(19) United States
(12) Patent Application Publication
(10) Pub. No.: US 2022/0280429 A1
(43) Pub. Date: Sep. 8, 2022

(54) Tunable leukocyte-based biomimetic nanoparticles and methods of use

(71) Applicant: The Methodist Hospital, Houston, TX (US)

(72) Inventors: Assaf Yosef Zinger, Houston, TX (US); Francesca Taraballi, Houston, TX (US)

(21) Appl. No.: 17/803,152

(22) Filed: Mar. 3, 2022

Related U.S. Application Data
(60) Provisional application No. 63/156,319, filed on Mar. 3, 2021.

(51) Int. Cl. A61K 9/127; A61K 45/06

(52) U.S. Cl. CPC A61K 9/1277; A61K 45/06; B82Y 5/00

(57) ABSTRACT

Disclosed are liposomal formulations and biomimetic proteolipid nanoparticles that possess remarkable properties for targeting compounds of interest to particular mammalian cell and tissue types. Leukocyte-based biomimetic nanoparticles are disclosed that incorporate cell membrane proteins to transfer the natural tropism of leukocytes to the final delivery platform. However, tuning the protein integration can affect the in vivo behavior of these nanoparticles and alter their efficacy. Here it is shown that, while increasing the protein:lipid ratio to a maximum of 1:20 (wt./wt.) maintained the nanoparticle's structural properties, increasing protein content resulted in improved targeting of inflamed endothelium in two different animal models. The combined use of a microfluidic, bottom-up approach, and the tuning of key synthesis parameters enabled the synthesis of reproducible, biomimetic nanoparticles for the improved targeted nanodelivery of a variety of inflammatory-based conditions, including particular cancers such as human breast cancer, and TNBC, in particular.

### CROSS-REFERENCE TO RELATED APPLICATIONS

The present application claims priority to U.S. Provisional Patent Appl. No. 63/156,319, filed Mar. 3, 2021 (expired); the contents of which is specifically incorporated herein in its entirety by express reference thereto.

### STATEMENT REGARDING FEDERALLY SPONSORED RESEARCH OR DEVELOPMENT

This invention was made with government support under Grant Nos. 1R56-CA213859 and F31-CA232705 awarded by the National Institutes of Health. The government has certain rights in the invention.

### NAMES OF THE PARTIES TO A JOINT RESEARCH AGREEMENT

Not Applicable.

### BACKGROUND OF THE INVENTION

### Field of the Invention

The present invention relates generally to the field of medicine, and in particular, to drug delivery compositions and formulations thereof. Disclosed are liposomal formulations and biomimetic proteolipid nanoparticles that possess remarkable properties for targeting compounds of interest to particular mammalian cell and tissue types. In particular embodiments, tunable leukocyte-based biomimetic nanoparticles are disclosed, which are highly suited for targeted drug delivery to selected mammalian cells in vitro and in situ and are particularly suited for the delivery of one or more therapeutic and/or diagnostic compositions to human triple-negative breast cancer patients.

### Description of Related Art

Nanoparticles (NP) represent a broad range of drug delivery vehicles that offer the ability to target diseased sites while minimizing off-target effects.¹ However, the complex biological milieu encountered by NP upon entry into the bloodstream poses significant biological barriers that thwart their ability to deliver their payload to the target tissue.² For example, systemic administration of NP exposes them to rapid uptake and clearance by components of the mononuclear phagocyte system (MPS).³ As a result, these NP do not reach the target site and, thereby, do not exert their therapeutic effects.

Previous efforts to overcome these challenges have included the incorporation of polyethylene glycol (PEG) to improve circulation times⁴ and conjugation of targeting moieties, such as antibodies and peptides⁵, to facilitate preferential accumulation to disease sites. Over time, increasing evidence highlighted the limitations of these strategies, such as the immune response to repeated injections of PEG and the high variability in conjugation densities of targeting moieties on NP surface.^{6,7}

Biomimetic NP represent an emerging class of NP for addressing the current challenges faced by the field of nanomedicine through biomimicry of native cells.^{8,9} Work in this field encompasses a broad range of NP, ranging from those mimicking red blood cells¹⁰ to immune cells¹¹ to even cancer cells.¹² Use of these biomimetic approaches has shown how traditionally used NP platforms can now harness the features of native cells to achieve specific function while maintaining the superior delivery capabilities of a synthetic NP.^{13,14} Examples of this include red blood cell membrane-coated polymeric NP that achieve longer circulation times for toxin removal in the blood¹⁵ and chemotherapy-loaded NP cloaked with cancer cell membranes for homotypic targeting of tumor cells.¹⁶

The syntheses of biomimetic NP have taken on two primary forms: top-down and bottom-up approaches. Isolation of whole cell membranes which are then applied in toto onto a synthetic NP core is an example of a top-down approach where the extracted component maintains the full biological complexity of the source.¹³ In contrast, bottom-up approaches utilize incorporation of ligands or other components as the building blocks to integrate into the final NP,¹⁶ such as the integration of membrane proteins into synthetic NP.¹⁷ While top-down approaches serve as a bridge between synthetic NP and source cells, bottom-up approaches offer more control in the tuning of the final NP formulation.^{17,18} Regardless of the synthesis approach utilized, maintenance of key NP physicochemical and biological characteristics, both during and after the synthesis process, is a crucial component in the engineering of these platforms.¹⁹ Achievement of specific functionality using these complex biomimetic NP warrants the careful and rational tuning of parameters associated with the synthesis process. Parameters such as the ratio of NP to extracted cell membrane, temperature used during the synthesis steps and post-synthesis purification process are examples of factors that must be carefully considered. The engineering of these design criteria has significant effects not only on the physicochemical properties of the NP, but also their biomimetic behavior under biological conditions.

Leukocyte-based biomimetic NP for targeting inflamed tissues (i.e., Leukosomes) have been previously reported.¹⁷ Leukosomes have demonstrated the ability to home to sites of inflammation and preferentially adhere to inflamed endothelia.²² Previously, the feasibility of synthesizing these NP using two synthesis methods—thin layer evaporation and a microfluidic-based approach was demonstrated.^{17,19} Upon synthesis, characterization of the NP verified their physiochemical properties while their biological functions were demonstrated in a local inflammation model. As inflamed endothelia are a common feature in a large number of disease conditions (e.g., tumor,²³ sepsis,²⁴ traumatic brain injury,²⁵ atherosclerosis,²⁶ etc.), this NP platform provides a very powerful tool for effective targeting and therapeutic cargo delivery. Furthermore, the tunability of this targeting is important for the tailoring of these NP to a specific disease condition.

Building off this foundational work, this study demonstrates the tunability of this system within the context of inflammation. In particular, the engineering of the synthesis parameters by establishing key design criteria was performed. These design criteria included thresholds on size and PDI, conservation of key leukocyte proteins, maintenance of the lipid bilayer structure and NP stability. Recognizing the need for ease of scalability and translational strategies for NP synthesis, the microfluidic-based approach was used for synthesis of the NPs in this disclosure.

As the integration of proteins dictates the biological behavior of leukosomes, the inventors sought to modulate this behavior by optimizing the protein:lipid (P:L) ratio utilized in the synthesis process. Therefore, it was hypothesized that an increase in the protein content on the NP will be directly correlated to their biomimetic targeting function in vitro and in vivo.

To this end, the effects of varying the P:L ratio of the leukosomes were assessed while using a microfluidic-based, bottom-up NP synthesis process. Preservation and stability of key physiochemical (e.g., size, zeta potential, NP concentration and morphology) and biomimetic (e.g., protein integration and presence of key leukocyte biological markers) parameters were first evaluated. Then, to assess the short and long-term stability of these biomimetic NP, we assessed the changes in each of the aforementioned parameters over the duration of 21 days. From here, the different formulations were tested for in vitro targeting to inflamed endothelial cells, which are the most relevant cell population implicated in the innate targeting of leukocytes to sites of inflammation.²⁷ Furthermore, preferential accumulation to sites of inflammation within the disease context was studied using murine lipopolysaccharide-induced local inflammation (LLI) and triple negative breast cancer (TNBC) in vivo models.

An improved understanding of how tuning this biomimetic NP's targeting capabilities is vital for future therapeutic applications of this platform. Enhancing this behavior by increasing the P:L ratio while using native leukocyte membrane proteins encompasses a simple but powerful approach. What results is a reproducible, potent biomimetic NP formulation that specifically targets the site of inflammation while reducing off-target effects on healthy tissues.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1 is a schematic of the different protein:lipid ratios (P:L) biomimetic NP microfluidic synthesis, characterization and in vitro and in vivo experiments. (1) Leukocytes (J774 cell line) were cultivated in vitro and used to extract (2) membrane proteins for the synthesis of (3) different P:L nanoparticles (NP) using a microfluidic approach by increasing membrane protein concentration in the aqueous phase. For Lipo, no membrane proteins were added; while, we added 0.058 mg/mL of protein extract for Leuko1:100; 0.145 mg/mL for Leuko1:40 and 0.29 mg/mL for Leuko 1:20. The physical, chemical and biological properties of the NP were characterized (4) and then tested in vitro using inflamed endothelial cells (5). Finally, the enhanced targeting was evaluated in vivo using LPS-induced local inflammation (LLI) and triple-negative breast cancer (TNBC) models. Illustration created using Biorender.com;

FIG. 2A, FIG. 2B, FIG. 2C, FIG. 2D, FIG. 2E, FIG. 2F, and FIG. 2G show the physiochemical and biomimetic properties characterization of NP. Physicochemical characterization of biomimetic NP showed increasing NP protein content decreased zeta potential and no effects on the size, polydispersity index (PDI), concentration and morphology. Multiple NP formulations of liposomes, Leuko1:100, Leuko1:40 and Leuko1:20 were assessed for their (FIG. 2A) size, (FIG. 2B) PDI, (FIG. 2C) concentration and (FIG. 2D) zeta potential (N=4). Lipo, Leuko1:100, Leuko1:40 and Leuko1:20 were imaged by cryo-TEM (FIG. 2E) and found to have similar lipid bilayer morphology. Scale bars=50 nm. SDS-Page gel (FIG. 2F) and Western blots (FIG. 2G) for five leukocyte membrane proteins markers (CD11b, CD18, CD45, CD47 and CD11a) indicates increasing protein gradient as more membrane proteins were added during the synthesis step. Results are shown as mean±SEM. One-way ANOVA followed by Tukey's multiple comparison test was used to determine statistical probabilities. P value ≤0.05 among means was considered as statistically-significant;

FIG. 3A, FIG. 3B, and FIG. 3C illustrate the in vitro toxicity and uptake of biomimetic NP in inflamed endothelial cells. Increasing protein content on NP resulted in no cytotoxicity and increased association and uptake by inflamed endothelial cells. (FIG. 3A) Endothelial cells were incubated with NP and toxicity evaluated by MTT assay. Cell viability after 24 h showed no significant decreases due to NP. NP association by inflamed endothelial cells (FIG. 3B) was confirmed by flow cytometry. Relative uptake, as measured by mean fluorescence intensity normalized to the liposomes treated cells, increased with increasing protein content on NP. Inflamed endothelial uptake of fluorescent NP (red) were also visualized by Z-stack confocal imaging (FIG. 3C). Following a 1-hr incubation, Leuko1:20 demonstrated significantly higher uptake across all NP formulations. Endothelial cells were stained for nuclei (blue) and cell membrane (green). Macro scale bar=100 μm, micro scale bar=27 μm. Results are shown as mean f SEM. Either one-way (FIG. 3A) or two-way (FIG. 3B) ANOVA followed by Tukey's multiple comparison test was used to determine statistical probabilities. P value ≤0.05 among means was considered as statistically significant;

FIG. 4A, FIG. 4B, FIG. 4C, FIG. 4D, FIG. 4E, FIG. 4F, FIG. 4G, FIG. 4H, FIG. 4I, and FIG. 4J demonstrate the in vivo targeting of NP in two inflammation models. Increasing protein content on NP resulted in increased targeting in LPS-induced local inflammation (LLI) and triple-negative breast cancer (TNBC). (FIG. 4A) LLI and (FIG. 4F) TNBC diseased mice were treated with NP as shown. Inflamed right ear (FIG. 4B) and tumor (FIG. 4G) targeting of fluorescent NP were imaged by In vivo Imaging System (IVIS). Following 3-5 hr from systemic administration, Leuko1:20 demonstrated significantly higher targeting in both in vivo models (FIG. 4C and FIG. 4H) across all NP formulations. These results were also verified using ex vivo analysis for both targeted organs (FIG. 4D and FIG. 4I). Results are shown as mean±SEM. Either one-way (FIG. 4E and FIG. 4J) or two-way ANOVA (FIG. 4C, and FIG. 4H) followed by Tukey's multiple comparison test was used to determine statistical probabilities. P value ≤0.05 among means was considered as statistically-significant;

FIG. 5A, FIG. 5B and FIG. 5C show in vivo biodistribution and safety of biomimetic NP. Increasing protein content on NP were well tolerated and resulted in different biodistribution in vivo. Mice were administrated with NP groups via IV injection. Mice were euthanized after either 8 h (LLI model) (FIG. 5A) or 24 hr (TNBC model) (FIG. 5B), organs collected and imaged. Tissue sections of liver, spleen, lung, and kidney were prepared for H&E staining (FIG. 5C). Scale bar=100 μm. Results are shown as mean±SEM. One-way ANOVA followed by Tukey's multiple comparison test was used to determine statistical probabilities. P value ≤0.05 among means was considered as statistically-significant;

FIG. 6 shows the effect of protein buffer on NP size. Increasing volume of protein buffer in aqueous phase of the NP synthesis resulted in an associated increase of NP size, with 180 μL being the maximum volume to be used to maintain a NP size of 200 nm. Results are shown as mean±SEM, N=3. Unpaired, two-tailed t-test was used to determine significant difference between 180 μL to 200 μL. A P-value ≤0.05 was determined to be statistically-significant;

FIG. 7A, FIG. 7B, and FIG. 7C show the native protein:lipid ratio. Combining empirical and mathematical calculations, the native P:L ratio of the cells used to synthesize particles was calculated to be 0.01. FIG. 7A) Quantification of proteins (left) and lipids (right) of J774 cells. FIG. 7B) Schematic highlighting the size and component differences of cells and NP. FIG. 7C) Table outlining the calculated values based on the quantification from (FIG. 7A) and scaling factor calculated based on the surface area of cells and NP;

FIG. 8A and FIG. 8B show NP biomimetic markers, original WB membranes and quantification; five leukocytes membrane proteins markers and one intracellular marker were characterized and quantified after the NP synthesis and purification (FIG. 8A). WB quantification indicates an increase in CD11a, CD11b, CD18, CD45 and CD47 quantity among the NP groups with no presence of intracellular protein marker, ACTB, or nuclear protein marker, NP62 (FIG. 8B).

FIG. 9A, FIG. 9B and FIG. 9C show the CD11b orientation studies revealed equal distribution of the cytoplasmic and exoplasmic parts. (FIG. 9A) CD11b orientation was studied by conjugating either exoplasmic (N) or cytoplasmic (C) antibodies (Ab) to the surface of 2.8 μm mice IgG compensation beads. The beads+Ab complex were blocked for 30 min at RT using 1% BSA solution to prevent nonspecific binding. Subsequently, fluorescent biomimetic NP were incubated with the beads+Ab complex for 1 hr followed by three times of centrifugation to remove any unreacted Ab. The beads+Ab+fluorescent biomimetic NP were then analyzed using a LSRFortessa cell analyzer to assess the correct membrane protein orientation (FIG. 9B). Equal distribution between the C:N terminal were assessed when we normalized the median florescence intensity of each group. (FIG. 9C) ˜25% of singlet gate events were assessed for each of the Leuko groups while only ˜0.1% of singlet gate events were assessed for the Lipo group. One-way ANOVA followed by Tukey's multiple comparison test was used to determine statistical probabilities. P value ≤0.05 among means was considered as statistically significant. In FIG. 9C, all groups of the leukosomes were statistically significant compared to the liposomes group (P value ≤0.01 at least);

FIG. 10A, FIG. 10B, FIG. 10C, FIG. 10D, and FIG. 10E illustrate 21-day NP storage stability studies. Physicochemical and biological characterization of NP over 21 days (MilliQ water, 4° C.) showed no significant changes in their physicochemical properties while Leuko1:20 maintained a higher protein content. Dynamic light scattering and NanoSight analysis of all NP formulations for (FIG. 10A) size, (FIG. 10B) PDI, (FIG. 10C) zeta potential and (FIG. 10D) concentration (N=4). SDS-Page gels for protein content on all NP formulations showed higher protein content with increasing protein incorporation on NP (FIG. 10E) (Day 0 SDS gel, (FIG. 10F). Results are shown as mean±SEM. Two-way ANOVA followed by Tukey's multiple comparison test was used to determine statistical probabilities. P value ≤0.05 among means was considered as statistically significant;

FIG. 11A and FIG. 11B show the gating strategy for flow cytometry analysis of NP uptake. (FIG. 11A) Gating for control cells (FIG. 11B) Gating for cells treated with biomimetic NP;

FIG. 12A and FIG. 12B show non-inflamed and LPS-inflamed endothelial cells. Non-inflamed (FIG. 12A) and LPS-inflamed (100 ng/mL) endothelial cells (FIG. 12B). Scale bars=50 μm;

FIG. 13A, FIG. 13B, and FIG. 13C show the in vitro association and uptake of biomimetic NP by non-inflamed murine endothelial cells. NP association by inflamed endothelial cells was confirmed by flow cytometry (FIG. 13A). Both metrics of relative uptake, as measured by median fluorescence intensity normalized to the liposomes treated cells and % of events from the singlets gate, increased with increasing protein content on NP. Non-inflamed endothelial uptake of fluorescent NP (red) was also visualized by Z-stack confocal imaging (FIG. 13C). Following a 1 h incubation, Leuko1:20 demonstrated significantly higher uptake across all NP formulations. Endothelial cells were stained for nuclei (blue) and cell membrane (green). Macro scale bar=50 μm, micro scale bar=27 μm. Results are shown as mean t SEM. Either One-way (FIG. 13A) or two-way (FIG. 13B) ANOVA followed by Tukey's multiple comparison test was used to determine statistical probabilities. P value ≤0.05 among means was considered as statistically significant. Confocal images were adjusted by +40% brightness ˜40% contrast;

FIG. 14 illustrates the percentage of positive cells in singlet gate of inflamed treated cells. The percentage of cells in the positive gate of the singlet gates was quantified. No significant differences were observed across the groups;

FIG. 15A and FIG. 15B show the ex vivo imaging of left and right ears for biomimetic NP targeting in a LLI model. Ex vivo imaging of both inflamed right (FIG. 15A) and non-inflamed left (FIG. 15B) ears of the LLI model mice demonstrated NP targeting only in the inflamed, right ears. The low signal detected in the left ears is attributed to the presence of blood in the lower part at site of the cut;

FIG. 16 illustrates TNBC tumor luminescence quantification. Mice were imaged 10 min post intraperitoneal injection of luciferin at 150 mg/kg. As no statistically significant difference was determined following quantification of the luminescence signal, tumors were deemed to be similar in size prior to administration of NP. Results are shown as mean±SEM. One-way ANOVA followed by Tukey's multiple comparison test was used to determine statistical probabilities. P value ≤0.05 among means was considered as statistically significant;

FIG. 17A and FIG. 17B depict in vivo biomimetic NP liver accumulation in TNBC model. In vivo images (FIG. 17A) and quantification (FIG. 17B) of NP fluorescence in the livers of TNBC tumor-bearing mice indicated the higher accumulation of Leuko1:20 for up to 8 hr when compared to the other NP groups. Results are shown as mean±SEM. Two-way ANOVA followed by Tukey's multiple comparison test was used to determine statistical probabilities. P value ≤0.05 among means was considered as statistically significant;

FIG. 18 shows the ex vivo imaging of TNBC tumors for NP accumulation after 24 hr. IVIS images of TNBC tumors at 24 hr confirmed the higher NP accumulation of Leuko1:20 when compared to other NP groups;

FIG. 19A, FIG. 19B, FIG. 19C, FIG. 19D, and FIG. 19E show intravital microscopy for NP accumulation in inflamed vasculature of TNBC mice. Leuko 1:20 demonstrated higher targeting to the inflamed blood vessels of the tumor 3 hr after systemic administration, the NP were imaged and assessed using intravital microscopy. Lipo, (FIG. 19B) Leuko1:100, (FIG. 19C) Leuko1:40, and (FIG. 19D) Leuko1:20. (FIG. 19E) Quantitative analysis of minimum 5 different locations in at least 3 mice for each group of NP. Scale bar=100 μm. Contrast was adjusted +20% for all images;

FIG. 20A, FIG. 20B, and FIG. 20C show the biomimetic NP accumulation in heart, lungs and spleen, respectively, in LLI model after 8 hr. Ex vivo imaging of heart (FIG. 20A), lungs (FIG. 20C) and spleen (FIG. 20C) of the LLI model mice. No major differences were observed in the NP accumulation in these organs;

FIG. 21A, FIG. 21B, and FIG. 21C show the biomimetic NP accumulation in liver, kidneys and blood, respectively, in LLI model after 8 hr. Ex vivo imaging of liver (FIG. 21A), kidneys (FIG. 21B) and blood (FIG. 21C) of the LLI model mice. Visual inspection revealed no major differences in NP accumulation in the liver and kidneys, while Leuko1:20 exhibited higher signal;

FIG. 22A, FIG. 22B, FIG. 22C, FIG. 22D, FIG. 22E and FIG. 22F show the biomimetic NP accumulation in heart, lungs, spleen, liver, kidneys and blood of TNBC tumor-bearing mice after 24 hr. Ex vivo imaging of heart (FIG. 22A) and lungs (FIG. 22B), spleen (FIG. 22C), liver (FIG. 22D), kidneys (FIG. 22E) and blood (FIG. 22F); and

FIG. 23A, FIG. 23B, FIG. 23C, FIG. 23D, FIG. 23E and FIG. 23F show the image analysis of collagen fibers workflow. FIG. 23A) One representative 4× image was chosen (FIG. 23B). For each image, three random 10× magnification images were chosen and the same scale was set for all (1.3 pixel/μm). (FIG. 23C) Masson's Trichrome color deconvolution plug-in was applied to each image and the green channel was chosen for the quantification of collagen fibers. (FIG. 23D) The same threshold (min value was 235 and max value was 255) was applied to each image. (FIG. 23E) A random ROI of the same size was generated, and quantification performed on 4 ROIs for each single image.¹ (FIG. 23F) Quantification of collagen fibers revealed an increase in fibrosis following injection of LPS and no further increase observed in the NP injected mice.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

### Pharmaceutical Formulations

In certain embodiments, the present invention concerns nanovesicle compositions prepared in pharmaceutically-acceptable formulations for delivery to one or more cells or tissues of an animal, either alone, or in combination with one or more other modalities of diagnosis, prophylaxis and/or therapy. The formulation of pharmaceutically-acceptable excipients and carrier solutions is well known to those of ordinary skill in the art, as is the development of suitable surgical implantation methods for using the particular membrane compositions described herein in a variety of treatment regimens, and particularly those involving treatment of mammalian cancers such as human breast cancer, and TNBC-type cancers in particular. Sterile injectable formulations may be prepared by incorporating the disclosed leukosome-based drug delivery compositions in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The leukosome-based drug delivery compositions disclosed herein may also be formulated in solutions comprising a neutral or salt form to maintain the integrity of the vesicles prior to administration.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, implantation regimen, formulation, and preparation of the leukosome-based drug delivery compositions disclosed herein will be within the purview of the ordinary-skilled artisan having benefit of the present teaching. It is likely, however, that the administration of a particular leukosome composition may be achieved by a single administration to provide the desired benefit to the patient undergoing such a procedure. Alternatively, in some circumstances, it may be desirable to provide multiple, or successive administrations of the leukosome-based agents, either over a relatively short, or even a relatively prolonged period, as may be determined by the medical practitioner overseeing the individual undergoing treatment.

The leukosome-based drug delivery compositions disclosed herein are not in any way limited to use only in humans, or even to primates, or mammals. In certain embodiments, the methods and leukosome-based drug delivery compositions disclosed herein may be employed in the treatment of avian, amphibian, reptilian, and/or other animal species, and may be formulated for veterinary surgical use, including, without limitation, for administration to selected livestock, exotic or domesticated animals, companion animals (including pets and such like), non-human primates, as well as zoological or otherwise captive specimens, and such like.

The present invention overcomes limitations in the prior art by providing methods for the synthesis of biomimetic nanovesicles, termed leukosomes, which employ proteins derived from leukocyte plasma membranes integrated into a synthetic biocompatible phospholipid bilayer as a new drug delivery platform. In particular embodiments, a microfluidics-based synthesis is disclosed for preparation of populations of biomimetic nanovesicles having desired biochemical properties. The leukosomes synthesized and described herein provide improved drug delivery vehicles, which combine the cell properties of leukocytes and the drug-delivery features of liposomes to produce a highly desirable method of drug delivery to mammalian cells and tissues.

In particular embodiments, the biomimetic nanovesicles of the present disclosure have been employed to provide combinational therapy of therapeutic molecules such as siRNAs treat one or more forms of inflammation, or, when combined with one or more chemotherapeutics, to treat one or more types of cancer in an affected individual. These nanovector-based drug delivery compositions improve the accumulation of conventional drugs in selected mammalian tissues, and achieve better therapeutic effect over the available conventional therapies.

The present disclosure provides in one aspect, biomimetic nanovesicles that improve stability and loading doses for mammalian administration.

These nanovesicles can be used to preferentially target particular cell types, reduce phlogosis in a localized model of inflammation, while retaining both the versatility and physicochmiecal properties typical of conventional nanovesicle formulations.

### Chemotherapeutic Methods and Use

An important aspect of the present disclosure concerns methods for using the disclosed drug delivery formulations for treating or ameliorating the symptoms of one or more forms of inflammation, or cancer, including, for example, a metastatic cancer, such as melanoma metastasis to the mammalian lung. Such methods generally involve administering to a mammal (and in particular, to a human in need thereof), one or more of the disclosed drug delivery systems comprising at least a first anti-inflammatory and/or an anticancer composition, in an amount and for a time sufficient to treat (or, alternatively ameliorate one or more symptoms of) the identified inflammation and/or cancer in an affected mammal.

In certain embodiments, the nanovesicle formulations described herein may be provided to the animal in a single treatment modality (either as a single administration, or alternatively, in multiple administrations over a period of from several hours (hrs) to several days (or even several weeks or several months) as needed to treat the particular disease, disorder, dysfunction, or abnormal condition. Alternatively, in some embodiments, it may be desirable to continue the treatment, or to include it in combination with one or more additional modes of therapy, for a period of several months or longer. In other embodiments, it may be desirable to provide the therapy in combination with one or more conventional treatment regimens.

The present disclosure also provides for the use of one or more of the disclosed nanovesicle drug delivery compositions in the manufacture of a medicament for therapy and/or for the amelioration of one or more symptoms of inflammation and/or cancer, and particularly for use in the manufacture of a medicament for treating and/or ameliorating one or more symptoms of a mammalian inflammation and/or cancer, including, for example human diseases and disorders.

The present invention also provides for the use of one or more of the disclosed drug delivery nanovesicle formulations in the manufacture of a medicament for the treatment of a disease or disorder in a mammal, and in particular, for the treatment of one or more human diseases such as inflammation and/or cellular hyperproliferation (i.e., cancer).

### Therapeutic Kits

Therapeutic kits including one or more of the disclosed nanovesicle drug delivery compositions and instructions for using the kit in a particular treatment modality also represent preferred aspects of the present disclosure. These kits may further optionally include one or more additional therapeutic compounds, one or more diagnostic reagents, or any combination thereof.

The kits of the invention may be packaged for commercial distribution, and may further optionally include one or more delivery devices adapted to deliver the micro/nano composite drug delivery composition(s) to an animal (e.g., syringes, injectables, and the like). Such kits typically include at least one vial, test tube, flask, bottle, syringe, or other container, into which the micro/nano composite drug delivery composition(s) may be placed, and preferably suitably aliquotted. Where a second pharmaceutical is also provided, the kit may also contain a second distinct container into which this second composition may be placed. Alternatively, the plurality of leukosomes disclosed herein may be prepared in a single mixture, such as a suspension or solution, and may be packaged in a single container, such as a vial, flask, syringe, catheter, cannula, bottle, or other suitable single container.

The kits of the present invention may also typically include a retention mechanism adapted to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

While systemic administration is contemplated to be effective in many embodiments of the invention, it is also contemplated that formulations disclosed herein be suitable for direct injection into one or more organs, tissues, or cell types in the body. Direct organ administration of the disclosed leukosome compositions may be conducted using suitable means, including those known to those of ordinary skill in the oncological arts.

The pharmaceutical formulations of the leukosome compositions disclosed herein are not in any way limited to use only in humans, or even to primates, or mammals. In certain embodiments, the methods and drug delivery formulations disclosed herein may be employed using avian, amphibian, reptilian, or other animal species. In preferred embodiments, however, the drug delivery compositions of the present invention are preferably formulated for administration to a mammal, and in particular, to humans, as part of an oncology regimen for treating one or more cancers. The leukosome compositions disclosed herein may also be acceptable for veterinary administration, including, without limitation, to selected livestock, exotic or domesticated animals, companion animals (including pets and such like), non-human primates, as well as zoological or otherwise captive specimens, and the like.

Nanovesicle Compositions and Formulations Thereof

In certain embodiments, the present invention concerns nanovesicle compositions prepared in pharmaceutically-acceptable formulations for delivery to one or more cells or tissues of an animal, either alone, or in combination with one or more other modalities of diagnosis, prophylaxis and/or therapy. The formulation of pharmaceutically acceptable excipients and carrier solutions is well known to those of ordinary skill in the art, as is the development of suitable surgical implantation methods for using the particular membrane compositions described herein in a variety of treatment regimens, and particularly those involving bone regrowth.

Sterile injectable formulations may be prepared by incorporating the disclosed leukosome-based drug delivery compositions in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The leukosome-based drug delivery compositions disclosed herein may also be formulated in solutions comprising a neutral or salt form to maintain the integrity of the vesicles prior to administration.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like.

The amount, delivery regimen, formulation, and preparation of the leukosome-based drug delivery compositions disclosed herein will be within the purview of the ordinary-skilled artisan having benefit of the present teaching. It is likely, however, that the administration of a particular leukosome composition may be achieved by a single administration to provide the desired benefit to the patient undergoing such a procedure. Alternatively, in some circumstances, it may be desirable to provide multiple, or successive administrations of the leukosome-based agents, either over a relatively short, or even a relatively prolonged period, as may be determined by the medical practitioner overseeing the individual undergoing treatment.

The leukosome-based drug delivery compositions disclosed herein are not in any way limited to use only in humans, or even to primates, or mammals. In certain embodiments, the methods and leukosome-based drug delivery compositions disclosed herein may be employed in the treatment of avian, amphibian, reptilian, and/or other animal species, and may be formulated for veterinary surgical use, including, without limitation, for administration to selected livestock, exotic or domesticated animals, companion animals (including pets and such like), non-human primates, as well as zoological or otherwise captive specimens, and such like.

### Exemplary Definitions

Unless defined otherwise, all technical and scientific terms used herein have the same meaning as commonly understood by one of ordinary skill in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

Although any methods and compositions similar or equivalent to those described herein can be used in the practice or testing of the present invention, the preferred methods, and compositions are described herein. For purposes of the present invention, the following terms are defined below for sake of clarity and ease of reference: In accordance with long standing patent law convention, the words “a” and “an,” when used in this application, including the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, a “biocompatible” material is a synthetic or natural material used to replace part of a living system or to function in intimate contact with living tissue. Biocompatible materials are intended to interface with biological systems to evaluate, treat, augment, or replace any tissue, organ, or function of the body. The biocompatible material has the ability to perform with an appropriate host response in a specific application and does not have toxic or injurious effects on biological systems. One example of a biocompatible material can be a biocompatible ceramic. As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

The term “effective amount,” as used herein, refers to an amount that is capable of treating or ameliorating a disease or condition or otherwise capable of producing an intended therapeutic effect.

The terms “for example” or “e.g.,” as used herein, are used merely by way of example, without limitation intended, and should not be construed as referring only those items explicitly enumerated in the specification.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to perform one or more of the methods of the present disclosure. Optionally, such kit may include one or more sets of instructions for use of the enclosed reagents, such as, for example, in a laboratory or clinical application.

As used herein, the term “patient” (also interchangeably referred to as “host” or “subject”), refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a patient may be any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, or any animal under the care of a veterinary or animal medical care practitioner.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof. Further examples of suitable acids include, but are not limited to, hydrochloric, ydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic, and benzenesulfonic acids. Salts derived from appropriate bases include, but are not limited to, alkali such as sodium and ammonia.

As used herein, “polymer” means a chemical compound or mixture of compounds formed by polymerization and including repeating structural units. Polymers may be constructed in multiple forms and compositions or combinations of compositions. As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about two to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer.

Polypeptides and proteins of the present invention also include polypeptides and proteins that are or have been post-translationally modified and include any sugar or other derivative(s) or conjugate(s) added to the backbone amino acid chain.

The term “subject,” as used herein, describes an organism, including mammals such as primates, to which treatment with one or more of the leukosome-based drug delivery compositions disclosed herein can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent. As used herein, “synthetic” shall mean that the material is not of a human or animal origin.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, an anti-inflammatory compound, a nucleolytic compound, a radioactive isotope, a receptor, a pro-drug activating enzyme, which may be naturally occurring, produced by synthetic or recombinant methods, or any combination thereof. Cytokines and related proteins may also be used as the therapeutic agent. Examples of such cytokines include, without limitation, lymphokines, monokines, peptide- and polypeptide-hormones and the like.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition.

In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### Example

The following example is included to demonstrate preferred embodiments of the invention. It should be appreciated by those of skill in the art that the techniques disclosed in the example that follows represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Enhancing Inflammation Targeting Using Tunable Leukocyte-Based Biomimetic Nanoparticle Compositions

### Materials and Methods

### Reagents

Membrane protein extraction kit, chloroform, methanol, Tween 20 and 2-Mercaptoethanol (Sigma Aldrich, St. Louis, Mo., USA). Dipalmitoylphosphatidylcholine (DPPC), 1,2-dioleoyl-sn-glycerol-3-phosphocholine (DOPC) and cholesterol (ovine wool, >98%) (Avanti Polar Lipids, Inc, Alabaster, USA). Float-A-Lyzer™ G2 dialysis devices (Spectrum™ Labs, MA, USA). Phosphate Buffered Saline (PBS) 10× solution, Syringe Filters 0.22 μm sterile PVDF, Millipore Sigma™ Milli-Q™ Ultrapure Water Systems Accessory, and Pierce™ Rapid Gold BCA Protein Assay Kit (Fisher Scientific, PA, USA). Dynamic Light Scattering (DLS), NanoSight NS300 and disposable cuvettes primarily for the measurement of zeta potential (Malvern, Instruments, Worcestershire, United Kingdom). Semi microvolume disposable polystyrene cuvettes for size measurements, 10×Tris Buffered Saline (TBS), 10×Tris/Glycine/SDS, Precision Plus Protein™ Dual Color Standards, 10% Mini-PROTEAN® TGX™ Precast Protein Gels, Trans-Blot Turbo Mini Nitrocellulose, 2× Laemmli Sample Buffer and Clarity™ Western ECL Substrate (Bio-Rad Laboratories, CA, USA). Sodium Dodecyl Sulfate (SDS) (GE Healthcare, IL, USA). Antibodies for western blot (rabbit anti-CD11a (LS-C331613), rat anti-CD11b (MAB11241), goat anti-CD18 (AF2618), rabbit anti-CD45 (EPR20033), goat anti-CD47 (ab108415), mouse anti-ACTB (A5441), mouse anti-NP62(sc-48389), anti-rabbit IgG-HRP, anti-goat IgG-HRP, anti-mouse IgG-HRP and anti-rat IgG (Bio-Techne Corporation, Minnesota, United States). FLUOstar Omega microplate reader (BMG, Labtech Ortenberg, Germany). NanoAssemblr™ Benchtop and Microfluidic Cartridge (Precision Nanosystems, Vancouver, Canada). Gelatin coating solution (Cell Biologics, Chicago Ill.).

### Cell Lines

J774 murine macrophages were purchased from ATCC and cultured in DMEM high glucose complete media supplemented with 1% L-glutamine and 1% Penstrep. BALB/c murine endothelial cells were purchased from Cell Biologics and cultured in complete murine endothelial media (Cell Biologics, Chicago, Ill., USA).

### Membrane Protein Extraction and Quantification

MP were extracted from J774 cells using a ProteoExtract® Native Membrane protein extraction kit and subsequently quantified for their concentration. Briefly, J774 cells were resuspended in 2 ml of wash buffer and centrifuged twice at 4° C., 300×g for 10 min. Afterward, the pellet was resuspended in 2 mL of buffer I with 10 μL Protease Inhibitor Cocktail and the sample was incubated at 4° C. for 10 min followed by centrifugation at 4° C., 16,000×g for 15 min. Subsequently, the pellet was resuspended in 1 mL of buffer II with 5 μL protease inhibitor cocktail and the sample was incubated at 4° C. for 30 min followed by a final centrifugation at 4° C., 16,000×g for 15 min. Lastly, the supernatant containing the MP was transferred to another tube and stored at −80° C. Quantification of the extracted protein concentration was determined using a Pierce™ Rapid Gold BCA Protein Assay kit. A calibration curve using albumin diluted in 1×PBS to following concentrations was prepared—0, 25, 125, 250, 500, 750, 1000 and 1500 μg/mL. MP and extraction buffer II were diluted in 1×PBS 1:5 (v/v). 20 μL of all samples were loaded in triplicate in a 96-well microplate and mixed with 200 μL of rapid Gold BCA reagent created by mixing reagents A and B 50:1 (v/v). The plate was then covered with aluminum foil and incubated for 10 min. The absorbance was measured at 480 nm with the plate reader.

### NP Synthesis

NP were synthesized using DPPC, DOPC and cholesterol (4:3:3 molar ratio) at a final lipid concentration of 9 mM. Lipids were dissolved in ethanol at 28 mg/ml, 22.5 mg/ml and 11 mg/mL, respectively. All stocks were agitated and sonicated for 5 minutes at 45° C. The starting volume of the lipid solution for liposomes, Leuko1:100 (protein:lipid (w/w)), Leuko1:40 (w/w) and Leuko1:20 (w/w) was 333.33 μL, 94.3 μL DPPC, 94.3 μL DOPC, 94.3 μL cholesterol and 50.4 μL ethanol, respectively. For the liposomes, aqueous buffer consisted of 667 μL MilliQ water. For all other NP formulations, the aqueous phase was comprised of MPs and MilliQ water at a final volume of 667 μL. Volume of MPs to be added was based on the protein:lipid ratio (w/w) for each NP formulation. As the lipid mass for 1 mL of formulation equated to 5.8 mg, the following amount of MPs were added: 0.058 mg for Leuko1:100, 0.145 mg for Leuko1:40 and 0.29 mg for Leuko1:20. The organic phase containing the lipids and the aqueous phase containing the MPs were heated at 45° C. for 3 and 1 min, respectively.

The microfluidic cartridge was first cleaned with 4 mL water (left inlet) and 4 mL ethanol (right inlet) and the following parameters were used: total volume 4 mL, flow ratio water:ethanol 1:1 (v/v), total flow ratio 4 mL/min, left and right syringe 5 mL, start waste 0.15 mL and end waste 0.05 mL. Next, the NP were synthesized by loading the aqueous phase to the left inlet and organic phase loaded to the right inlet of the cartridge using a 3-mL syringe (left inlet) and a 1-mL syringe (right inlet), using the following parameters: total volume 1 mL, flow ratio water:ethanol 2:1 (v/v), total flow ratio 1 ml/min, start waste 0.15 ml and end waste 0.05 ml.

### NP Purification to Remove Unbound Membrane Proteins

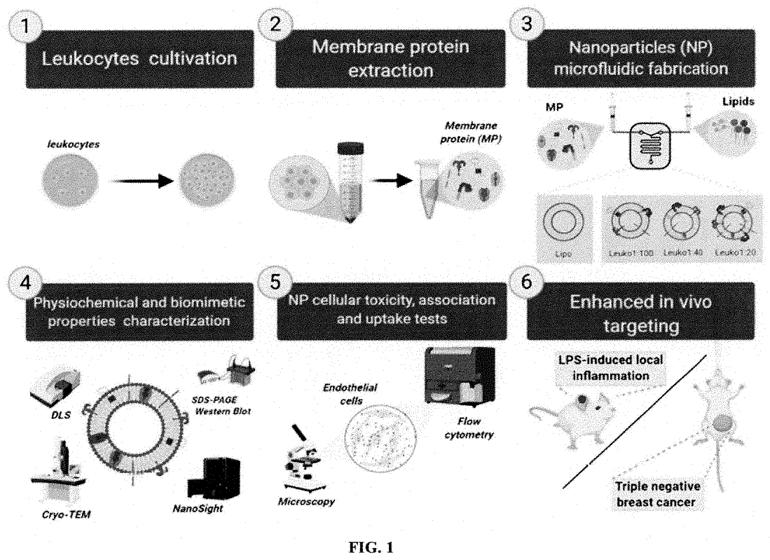

After step 2 (FIG. 1) the samples were loaded into a 1000 kDa Float-A-Lyzer and dialyzed separately in 1 L of MilliQ water for each formulation. The buffer was changed after 1 and 3 h and samples were collected after 19 h. Step 3 (FIG. 1) was performed under gentle stirring at 4° C. The samples were filtered using 0.22 m PVDF filters after 19 h.

Nanoparticles physiochemical properties characterization: size, PDI, zeta potential measurements and NP concentration and stability test NP characterization (size, polydispersity index (PDI), zeta potential (ZP) and NP concentration) was done. Briefly, size, PDI and ZP were evaluated using a Malvern Zetasizer (Malvern Panalytical, Westbourough Mass.). For size and PDI measurements, the samples were diluted in MilliQ water or 1×PBS 1:100 (v/v) and three measurements of 15 runs each were acquired. For ZP, samples were diluted in MilliQ water 1:100 (v/v) and three measurements of 10 runs each were acquired. The final value for each sample was obtained by taking the average of the three measurements. The NP concentration was evaluated using a NanoSight NS300 (Malvern Panalytical, Westbourough Mass., USA). The samples were diluted in MilliQ water 1:10000 (vol./vol.) using the following parameters: 25° C., screen gain 1, camera level 13, infusion rate 100, flow ratio 1 mL/min. For each sample, 5 measurements were acquired with a duration of 60 sec each. A detection threshold equal to 7 was used to calculate the final NP concentration. The NP were stored in MilliQ water at 4° C. and characterization was repeated after 1, 3, 14, and 21 days. It should be noted, however, that NP concentration was only measured after 1- and 21-days post-synthesis.

### Cryo-EM Sample Preparation

The various NP solutions were vitrified and imaged at the Baylor College of Medicine Cryo-Electron Microscopy Core Facility (BCM, Houston, Tex., USA). Quantifoil R2/1, Cu 200 mesh Holey Carbon grids were pretreated with a 45-sec air-glow discharge to make the carbon surface hydrophilic. Alongside these grids, Quantifoil R2/1 200 Cu+4 nm Thin Carbon grids were also glow discharged for 10 sec to test the efficacy of the added layer of continuous carbon with binding the NP. Vitrification was performed using a Vitrobot Mark IV (FEI, Hillsboro, Oreg.) operated at 18° C. and 100% humidity. Each grid had 3 ul of nanoparlicle sample applied to it and blotted for 1-3 sec before being immediately submerged in liquid ethane. The frozen grids were then transferred into a JEOL 3200FS microscope (JEOL) outfitted with a Gatan K2 Summit 4 k×4 k direct detector (Gatan, Pleasanton, Calif., USA) and a post-column energy filter set to 30 eV. Before imaging, the microscope was carefully aligned to prevent any beam induced aberrations or astigmatism that can negatively impact image quality. Images were collected at magnifications of 15,000× and 30,000× with respective pixel sizes of 2.392 and 1.232 angstroms. Images were collected using an exposure time of 1 sec with an approximate dose rate of ˜20e−/Å²/s per image.

Synthesis, Physicochemical and Biomimetic Characterization of Leukocyte-Based Biomimetic NP with Varying P:L Ratios

Prior to commencing the synthesis of the NP with varying P:L ratios, we established design parameters that were critical to determining the success of synthesis and the ability of the NP to achieve intended biological functions. To this end, we set the following measurement parameters: 1) Size less than 200 nm, 2) polydispersity index (PDI) less than 0.2, 3) Negative zeta potential following incorporation of proteins, 4) Maintenance of lipid bilayer structure even when more proteins are added, 5) Conservation of key leukocyte markers on the NP and 6) NP stability for 21 days.

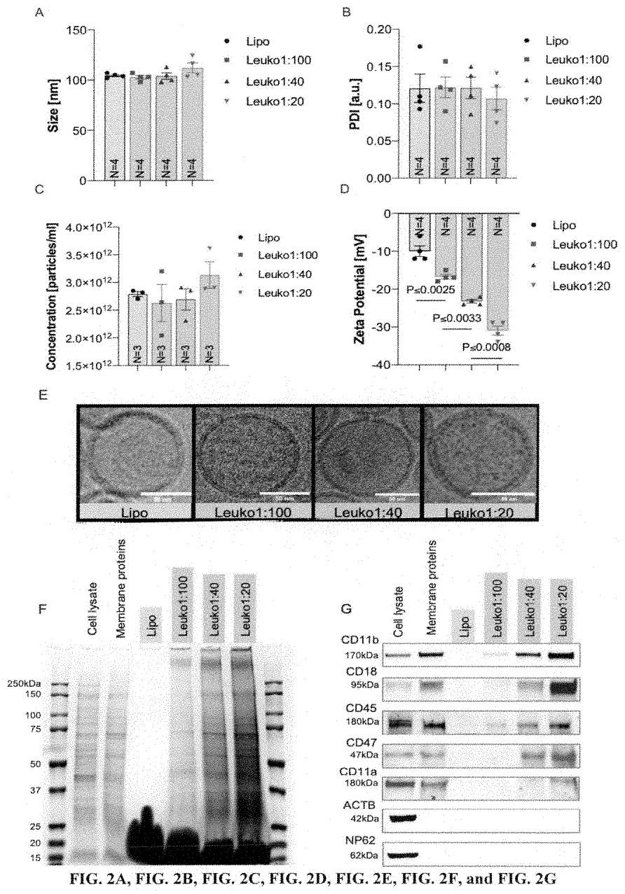

The synthesis of the NP is divided into two parts: extraction of membrane proteins from leukocytes followed by microfluidic synthesis of NP (FIG. 2A-FIG. 2G). Leukocytes were cultivated and membrane proteins extracted using a commercial kit.²⁸ Synthesis on the NanoAssemblr® involves feeding two phases, an organic phase containing lipids and an aqueous phase comprised of buffer with extracted membrane proteins, into the inlet ports of the chip. Mixing of these two phases within microstructures of the chip results in formation of the desired NP. One of the main steps during the microfluidic synthesis that differentiates the NP groups is the amount of membrane proteins added to the aqueous phase before NP assembly. The protein amount dictated the P:L ratio associated with each NP group: Lipo—none, Leuko1:100-0.058 mg/mL, Leuko1:40-0.145 mg/mL and Leuko1:20-0.29 mg/mL. The incorporation of different amounts of proteins in the NP structure, although a simple concept, was not trivial.

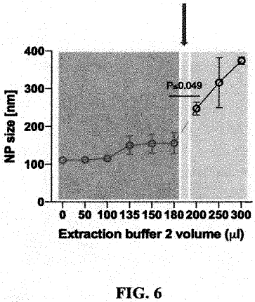

It was found that the proteins' suspension buffer dramatically affected the NP size and PDI. Thus, the inventors next determined the maximum amount of proteins' buffer that did not affect the desired physicochemical properties of the NP (FIG. 1). With these design criteria in mind, 180 μL of the protein buffer was determined to be the max volume that could be used, which corresponded to a maximum P:L ratio of 1:20. In addition, they also demonstrated how the P:L ratio tested with our NP formulations compares to the P:L ratio found on native leukocyte membranes (FIG. 6). Combining our empirical and mathematical calculations, the native P:L ratio was determined to be 1:100.

Using this microfluidic approach, we demonstrated that the different NP groups can be synthesized in a reproduceable manner (N=4), while maintaining the desired P:L ratio. The size and PDI of Lipo, Leuko1:100 and Leuko1:40 were relatively similar, with a size of 100 nm and PDI of 0.12 (FIG. 2A and FIG. 2B). Leuko1:20 NP were slightly larger in size (110 nm), but lower PDI at 0.1 (FIG. 2C). Particle concentration of the Lipo was 2.7×10¹² particles/mL, while Leuko1:100 and Leuko1:40 were around 2.5×10¹² particles/mL (FIG. 2D). Leuko1:20 had slightly larger particle concentration at 3×10¹² particles/mL (FIG. 2E). Moreover, we validated that increasing the membrane protein concentration during the NP assembly did not affect the NP size, PDI and concentration beyond the predetermined thresholds set in our design criteria (FIG. 2F). However, a gradient decrease in zeta potential (ZP) was observed as the membrane protein concentration was increased (FIG. 2G). More specifically, Leuko1:20 demonstrated a 3.1-fold decrease in ZP compared to Lipo, 1.5-fold decrease in ZP compared to Leuko1:100 and 1.3-fold decrease in ZP compared to Leuko1:40. Interestingly, when we visualized the NP using cryo-transmission electron microscopy (TEM), we noticed the NP also preserved their bilayer structure even after increasing the membrane protein concentration during the synthesis process (FIG. 2E).

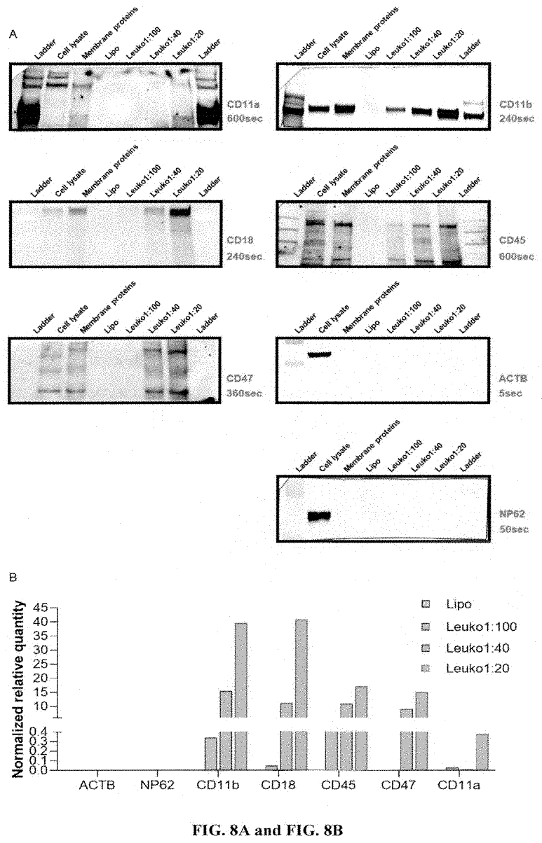

Following the physicochemical and structural studies, the NP were examined for their biomimetic properties (i.e. leukocytes' membrane protein content). SDS-PAGE analysis, revealed that membrane protein content was retained through the NP microfluidic synthesis. Moreover, we observed that the NP proteins' content exhibited a gradient increase with respect to increasing P:L ratio (FIG. 2F). Using western blots (WB), a similar gradient increase was observed for specific proteins: CD11b, CD18, CD45, CD47 and CD11a (FIG. 2G) and quantified (FIG. 8A and FIG. 8B). When compared to the Leuko1:100, Leuko1:40 and Leuko1:20 exhibited over a 37-fold and 87-fold increase in CD11b and CD18 integration, respectively (FIG. 8A and FIG. 8B). On the other hand, CD45 presence on the NP was 10- and 15-fold higher on the Leuko1:40 and Leuko1:20, respectively, when compared to the Leuko:100 (FIG. 2E).

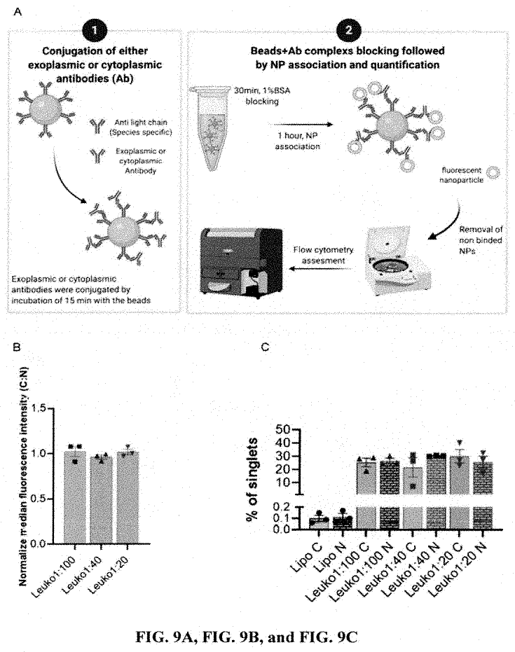

Notably, the presence of ACTB, an intracellular membrane protein marker and NP62, a nuclear protein marker, that served as negative controls for this experiment, were not detected among the NP samples. In an effort to understand the spatial orientation of the integrated proteins on the NP, we used flow cytometry while focusing on one target protein—CD11b (FIG. 9A). Orientation of the integrated protein was evaluated by whether the N-terminus (i.e. exoplasmic) or C-terminus (i.e. cytoplasmic) of the protein was exposed on the NP surface, using specific antibodies against the two termini of the same protein. The normalized quantification of the median fluorescence indicated no differences between N- or C-terminal orientation across all 3 NP groups (FIG. 9B). In addition, the percentage of positive events in the singlet gate for each of these termini was found to be approximately the same across all the NP groups (FIG. 9C). Taken together, this orientation study indicated that the integration of the CD11b protein occurs in a random manner, with an equal ratio of exposure of both the N- and C-termini on the NP surface.

### Characterization of Short and Long Storage Stability of NP Over 21 Days

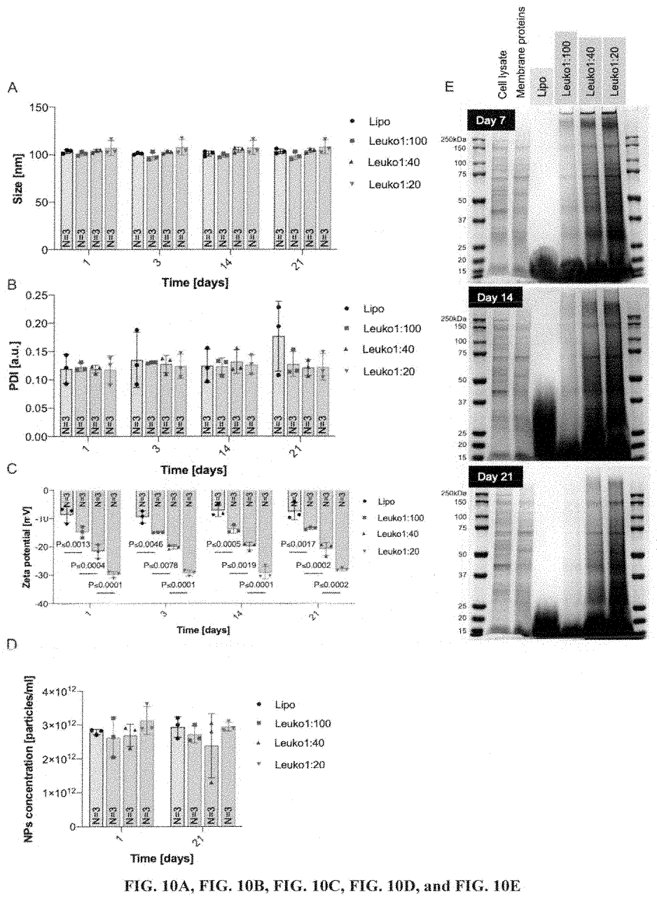

We then assessed both the structural and biomimetic stability of the NP when stored at 4° C. For structural stability tests, Dynamic Light Scattering (DLS) measurements for the NP size, PDI and ZP were done at both short and long timepoints post-synthesis: 1, 3, 14 and 21 days (FIG. 10A-FIG. 10E). NanoSight was used to assess the NP concentration changes after 21 days (FIG. 10A). For the biomimetic stability, SDS-PAGE was done every 7 days for 3 weeks in a row (FIG. 10B). We did not observe any statistically significant differences between the NP structural and biomimetic properties. In fact, no changes in size were observed across all the NP formulations over the 21-day period (FIG. 10C). Although the PDI of all the Leuko NP remained around 0.12, Lipo did exhibit an increase from 0.12 to 0.15 PDI at Day 21 (FIG. 10D). In addition, ZP remained the same in all the NP formulations (FIG. 10E).

Although particle concentration remained the same for Lipo, Leuko1:100 and Leuko1:20, the Leuko1:40 formulation showed a slight decrease of 0.5×1012 particles/mL by Day 21 (FIG. 10A). Lastly, SDS-Page gels indicated the maintained protein presence on the particles in all the Leuko formulations (FIG. 10D). Taken together, this suggested that the NP maintain their size, PDI, ZP and concentration while retaining the gradient increase in membrane protein content on the different biomimetic NP.

### In Vitro Toxicity and Uptake of Biomimetic Np by Inflamed Endothelial Cells

Given that the primary target of these NP upon systemic administration will be sites of inflammation, we chose to test uptake of the NP in vitro by the first inflamed cells they will encounter at these sites—endothelial cells. In particular, we inflamed murine endothelial cells with lipopolysaccharide (LPS), which also served as the basis of our LLI in vivo model.

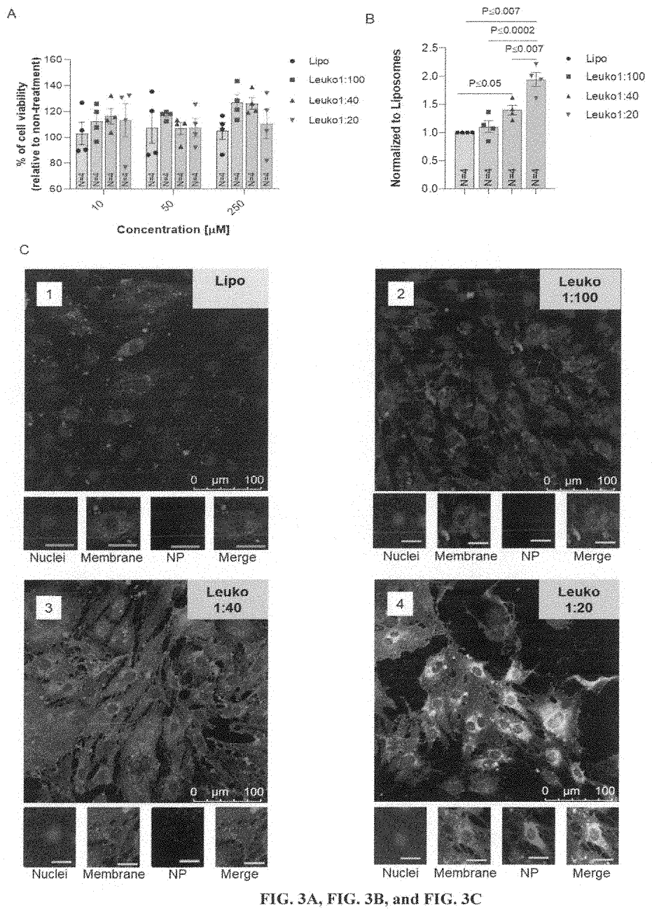

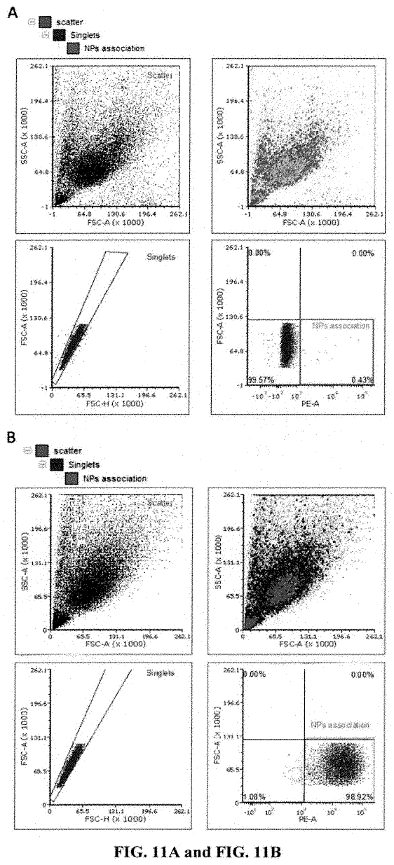

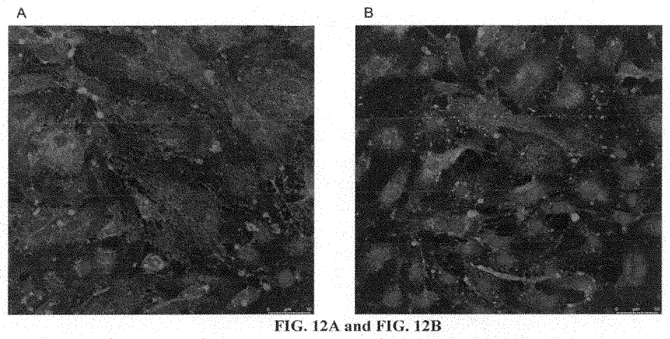

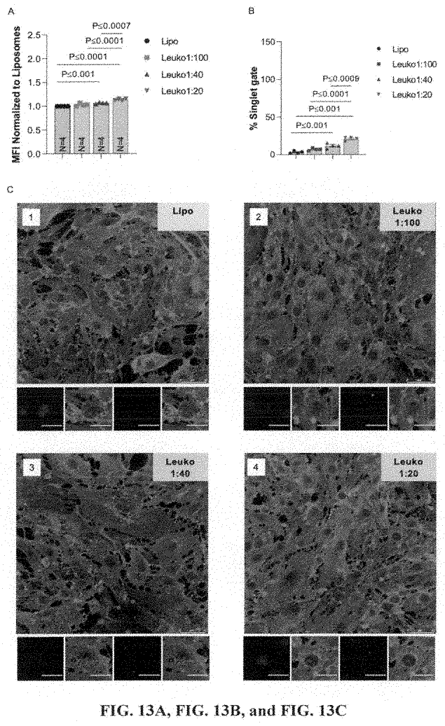

Prior to verifying the targeting abilities of the various NP formulations in vitro, we first confirmed that varying the protein concentration on the particles did not result in increased cytotoxicity. The incubation of NP with inflamed endothelial cells did not result in any cytotoxic effects after 24 h (FIG. 3A). The cells maintained 100% of viability for all tested NP concentrations. Then, the effect of the different P:L ratio on NP uptake was evaluated using both confocal imaging and flow cytometry for the quantification (gating strategy in FIG. 11A and FIG. 11B). Control images of non-inflamed and LPS-inflamed endothelial cells, along with NP treated non-inflamed cells, were also acquired for comparison (FIG. 12A, FIG. 12B, FIG. 13A, FIG. 13B, and FIG. 13C).

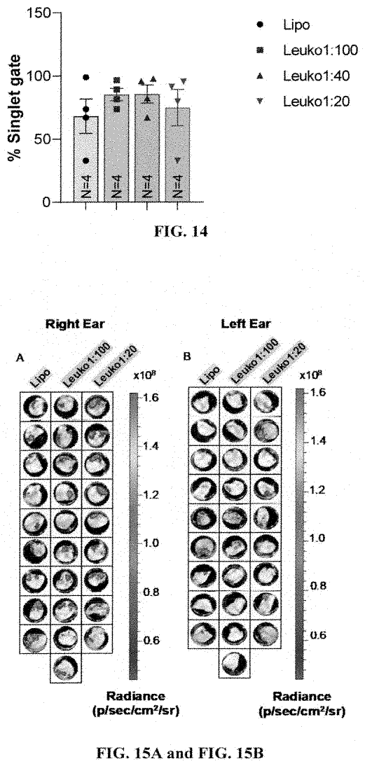

LPS-inflamed endothelial cells exhibited a gradient uptake pattern (FIG. 3B and FIG. 14) when evaluated by flow cytometry. More specifically, both Leuko1:40 and Leuko1:20 demonstrated significant uptake when compared to liposomes, with almost a 2-fold increase for the latter NP group. To further verify these observations, NP uptake by these cells was also visualized by confocal microscopy imaging. Once again, significant preferential uptake of the Leuko1:20 NP was evident while very little uptake was observed in the other NP groups (FIG. 3C).

### In Vivo Biomimetic NP Targeting in a LLI and TNBC Models

To examine the effects of varying the NP P:L ratio on their targeting of sites of inflammation, two different in vivo models were examined. Both a LLI model and a TNBC tumor model were chosen to investigate the biodistribution of the NP upon systemic administration. Once again, the strain of mice used for both models match the source of cells used for the NP synthesis. This was done to avoid immune responses associated with different cell sources.

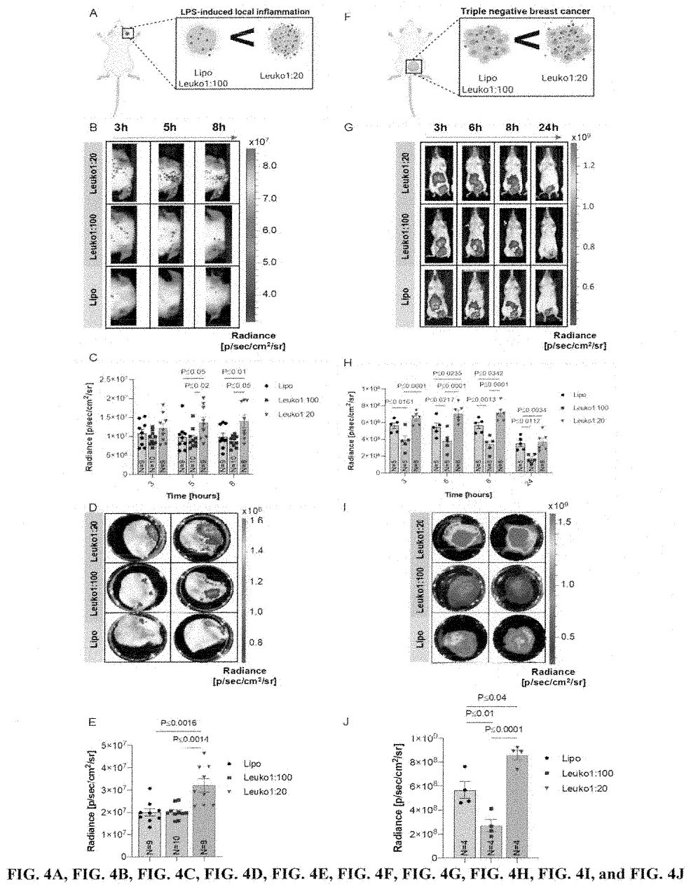

For the LLI model, mice were injected in one ear (right ear) with a single administration of LPS. Shortly after, mice were injected with the NP formulations via tail vein injection (FIG. 4A). Given the gradient behavior observed in our in vitro experiments, we chose to follow both biodistribution and target site accumulation of only the Lipo, Leuko1:100 and Leuko1:20 formulations. We recognized that these two Leuko groups represented the maximum and minimum thresholds of in vitro targeting and, thereby, providing us an opportune window to observe the differential patterns of accumulation between the NP groups. Within 5 hr of NP injection, significant differences could be observed in the NP accumulation in the inflamed ears only (right ears) (FIG. 4B). No sign of NP accumulation was noticed in the non-inflamed ears (left ear) among all NP groups. Leuko1:20 demonstrated a 1.5-fold increase in inflamed ear accumulation over both the Lipo and Leuko1:100 groups (FIG. 4C). This observed increase remained consistent also for the 8 h timepoint.

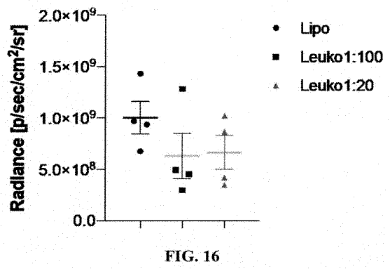

Ex vivo imaging of the ears demonstrated once more that NP accumulation could be observed only in inflamed ears, while control ears showed no NP accumulation (FIG. 4D, FIG. 15A and FIG. 15B). This further validated the ability of these biomimetic NP to specifically target the site of inflammation. In addition, the Leuko1:20 group exhibited a 1.75-fold increase in particle accumulation over the other NP formulations (FIG. 4E). While the LLI represented an acute inflammation model, we aimed to further validate the robust targeting abilities of these biomimetic NP in a tumor model, where inflammation stems from a host of underlying factors that emerge over time. Similar to the ear inflammation model, tumors were established in mice and verified by luminescence quantification to be similar in size prior to NP treatment (FIG. 16). Mice were then administered with the different NP formulations via tail vein injections (FIG. 4E). In vivo imaging of the whole mice indicated varying levels of accumulation in the tumor for up to 24 h (FIG. 4F).

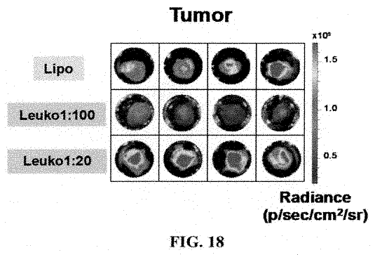

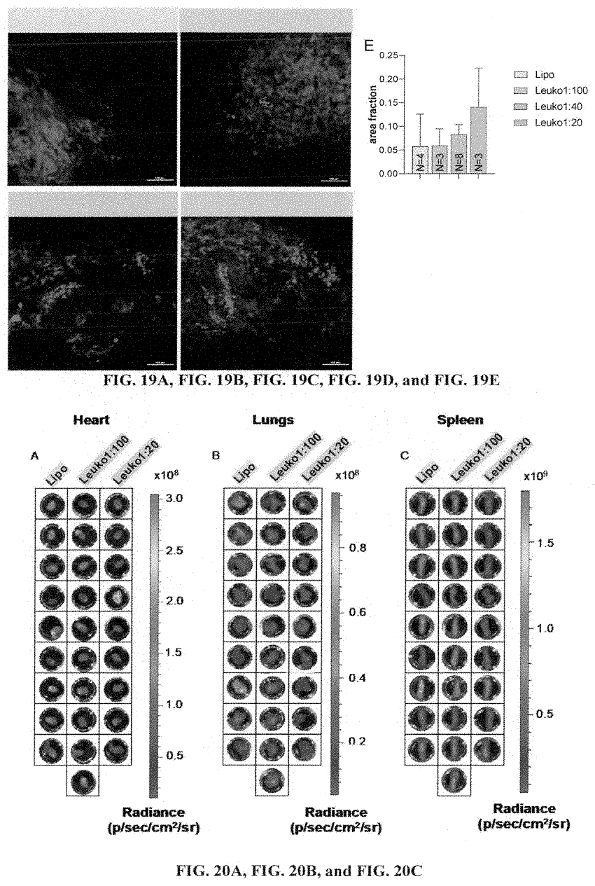

Significant differences in tumor accumulation could be observed between the Leuko1:20 and Lipo beginning at 6 h, while these differences could be seen at all timepoints when comparing the Leuko1:20 and the Leuko1:100 (FIG. 4G). Interestingly, the longer circulation time of the Leuko1:20 NP could also be observed, where the signal of liver accumulation in the Lipo group is reduced significantly by 6 h. In contrast, the Leuko1:20 group maintained a strong signal in the liver for up to 8 h (FIG. 4H). Ex vivo imaging at 24 h confirmed the superior accumulation of the Leuko1:20 NP in the tumor (FIG. 4I and FIG. 18). In fact, the Leuko1:20 exhibited a 1.4-fold and 2.7-fold increase in tumor accumulation over the Lipo and Leuko1:100 groups, respectively (FIG. 4J). In addition to this whole organ quantification for NP accumulation, intravital microscopy (IVM) was also performed on TNBC tumors, with imaging focused on the inflamed tumor vasculature. Quantification of NP signal within the vessels indicated that increasing protein content on the NP improved the targeting of the NP to the vasculature, with Leuko1:20 exhibiting 3-fold more averaged accumulation in the vessels over liposomes (FIG. 19A-FIG. 19E).

### SDS Gel and Western Blot Detection

After dialysis, NP were diluted with MilliQ water to a final lipid concentration of 6 mM. 150 μL of the samples were used for SDS gel and 300 μl (CD11b, CD18 detection), 600 μL (CD11a detection), 1200 μL (CD45, CD47 detection) and 3000 μL (ACTB, NP62 detection) for western blot. The samples were centrifuged at 4° C., 45000 rpm for 1 h. 2× sample buffer was prepared by mixing 2× Laemmli Sample Buffer with 2-Mercaptoethanol 20:1 (v/v). Then, the NP pellet for SDS gel was resuspended with 40 μl of 1× sample buffer (2× sample buffer mixed with water 1:1 (v/v)). For western blot, the pellet was resuspended with 2.5% SDS samples buffer (2× sample buffer mixed with 30% SDS and water) for CD11b and CD18, and 5% SDS samples buffer for CD11a, as the final concentration of SDS. For CD45, CD47, ACTB and NP62 detection, proteins in the pellets were purified and extracted with chloroform and methanol. Briefly, the NP pellets were resuspended with 50 μL of MilliQ water, followed by gently mixing with 400 ul of methanol and 100 uL of chloroform. After centrifugation at 15000 g for 2 min, 400 ul of methanol was added, then centrifuged again. The protein pellets were dried by removing the supernatant and the protein pellets were dissolved in the 1× sample buffer. Samples were denatured at 95° C. for 7 minutes.

For SDS gel, 5 μg MPs were resuspended in 1× sample buffer, while for the western blot, 10 μg (CD11b, CD18), 20 μg (CD11a) of MP were resuspended with each SDS sample buffer, and 40 μg (CD47) and 60 μg (CD45, ACTB, NP62) of MP were resuspended with 1× sample buffer. 10% Mini-PROTEAN® TGX™ Precast Protein Gel was used for both SDS gel and western blot and run on ice for approximately 2 h at 100 V. The SDS gel was washed three times for five minutes with MilliQ. Then, SimplyBlue™ SafeStain (Invitrogen, Carlsbad Calif.) was added, and the gel remained under constant agitation overnight at 4° C. Finally, the gel was washed with MilliQ water three times for ten minutes. To enhance the staining contrast, sodium chloride was added during the final wash. The analysis of the gel was done using the ChemiDoc XRS+ System. For the western blot, the gel was transferred to the membrane using the Trans-Blot Turbo Transfer System. The membrane was incubated under agitation at room temperature with 5% non-fat milk in 0.1% Tween 20 in TBS (TBST) for 1 hour.

Finally. the 5% non-fat milk was removed and MP markers (CD11a, CD11b, CD18, CD45, D47, ACTB and NP62) were detected by incubating the membrane using the following primary antibodies: rabbit anti-CD11a (LS-C331613) (diluted with milk 1:500 (v/v)), rat anti-CD11b (MAB11241) (diluted with milk 1:3000 (v/v)), goat anti-CD18 (AF2618) (diluted with milk 1:3000 (v/v)), rabbit anti-CD45 (EPR20033) (diluted with milk 1:1000 (v/v)), goat anti-CD47 (ab108415) (diluted with milk 1:1000 (v/v)), mouse anti-ACTB (A5441) (diluted with milk 1:1000 (v/v)) and mouse anti-NP62(sc-48389) (diluted with milk 1:100 (v/v)) in agitation at 4° C. overnight. After washing with TBST, the membranes were incubated under agitation at room temperature with the following secondary antibodies anti-rabbit IgG-HRP (diluted with milk 1:1000 (v/v)), anti-goat IgG-HRP (diluted with milk 1:1000 (v/v)), anti-rat IgG (diluted with milk 1:1000 (v/v)) and anti-mouse IgG (diluted with milk 1:1000 (v/v)) for 1 hour. Afterward, membranes were incubated in western ECL substrate using the Clarity Max Western Peroxide and the Clarity Max Western Luminol/Enhancer reagents 1:1 (v/v) for 5 minutes while covered with aluminum foil. Finally, MPs were detected using the ChemiDoc XRS+ System with the following exposures times: 600 sec for CD11a, 240 sec for CD11b, 240 sec for CD18, 600 sec for CD45, 360 sec for CD47, 5 sec for ACTB and 50 sec for NP62.

### NP Cytotoxicity

Prior to seeding, well plates were coated with gelatin coating solution, incubated for 30 min at 37° C. and excess coating solution removed. Murine endothelial cells were seeded in complete media in a 96-well plate at a seeding density of 8,000 cells/well. After 24 h, NP were resuspended in complete media and added to the cells at the following concentrations: 10, 50, 100, 250 and 500 μM. These concentrations were based on the lipid concentration of the NP after synthesis. Following a 24 h incubation with the NP, media was aspirated and replaced with MTT resuspended in completed media at a concentration of 0.5 mg/ml. After 2 h, MTT reagent was aspirated and replaced with equal volume DMSO. Following 30 minutes of gentle agitation at room temperature, absorbance was measured at 570 nm with reference wavelength of 630 nm.

### Confocal Imaging of Np Uptake

Prior to seeding, chamber slides were coated with gelatin coating solution, incubated for 30 min at 37° C. and excess coating solution removed. Murine endothelial cells were seeded in complete media in 8-well chamber slides at a seeding density of 10,000 cells/well. After 24 h, media was replaced with fresh media resuspended with LPS (Millipore Sigma, St. Louis Mo.) at a concentration of 100 ng/ml. Following a 24 h incubation with LPS, cells were washed with 1×PBS and treated with rhodamine-labeled NP resuspended in complete media at a concentration of 100 μM. After 1 hour, cells were washed with 1×PBS, fixed with 4% PFA, and stained with WGA-Alexa 488 (Invitrogen, Carlsbad Calif.) and DAPI. Slides were then imaged on a Leica.

### Flow Cytometry of Nanoparticle Uptake

Prior to seeding, well plates were coated with gelatin coating solution, incubated for 30 min at 37° C. and excess coating solution removed. Murine endothelial cells were seeded in complete media in 24-well plates at a seeding density of 50,000 cells/well. After 24 h, the media was replaced with fresh media resuspended with LPS at a concentration of 100 ng/ml. Following a 24 h incubation with LPS, cells were washed with 1×PBS and treated with rhodamine-labeled NP resuspended in complete media at a concentration of 100 uM. After 1 hour, cells were detached using TrypLExpress, spun down and washed with 1×PBS. Cells were collected into flow cytometry tubes and assayed on BD LSRII flow cytometer.

### In Vivo Targeting and Biodistribution Studies

All animal studies were performed in accordance with the guidelines of the Animal Welfare Act and the Guide for the Care and Use of Laboratory Animals approved by The Houston Methodist Institutional Animal Care and Use Committee guidelines (Houston, Tex., USA). LLI model was generated in BALB/c mice (6-8 weeks years old and 25 gr) (Charles River Laboratories, Wilmington, Mass., USA) by a one-time injection of LPS (50 μg) in the right ear. 100 μL of Cy5.5-labeled NP were administrated via tail vein injection 30 minutes after LPS administration. Mice were prepared for IVIS at 3, 6 and 8 hr after particle injection to assess targeting and biodistribution. IVIS image acquisition parameters were the following: Em=720, Ex=640, Epi-illumination, Bin:(HR)4, FOV:18.4, f2, 0.5 sec. After 8 hr, ears, heart, lungs, liver, spleen, kidneys and blood were collected and imaged on the IVIS. Quantification of IVIS images was performed using the Living Image software.

TNBC model was established by injecting a total of 3×10⁵ 4T1-Red-FLuc (PerkinElmer, Waltham, Mass., USA) cells, suspended in 50 μL of 1×PBS, subcutaneously into the mammary fat pad of 10-week-old BALB/c female mice (Charles River Laboratories, Wilmington, Mass., USA). Approximately 14 days following tumor cell injection, tumor size was verified using luminescence imaging prior to NP injection. Mice were injected intraperitoneally with luciferin (10 mg/kg) and imaged 10 min post-injection. 100 μL of Cy7-labeled NP were administrated via tail vein injection and animals imaged on IVIS after 3, 6, 8 and 24 hr. IVIS image acquisition parameters were the following: Em=820, Ex=745, Epi-illumination, Bin:(HR)4, FOV:18.4, f2, 1s. After 24 h, ears, heart, lungs, liver, spleen, kidneys and blood were collected and imaged on the IVIS. Quantification of IVIS images was performed using the Living Image software.

### Histological Sample Preparation and Imaging

Tissue samples from the lungs, spleen, liver, kidneys, and heart from the LLI mice were washed using 1×PBS and then fixed using 10% natural buffer formalin. Samples were stored at 4° C. for 24 hr before they were paraffin embedded, axial sectioned and hematoxylin and eosin (H&E) stained. Slide were imaged using Keyence BZ-X810 microscope.

### Additional Studies and Supplementary Information

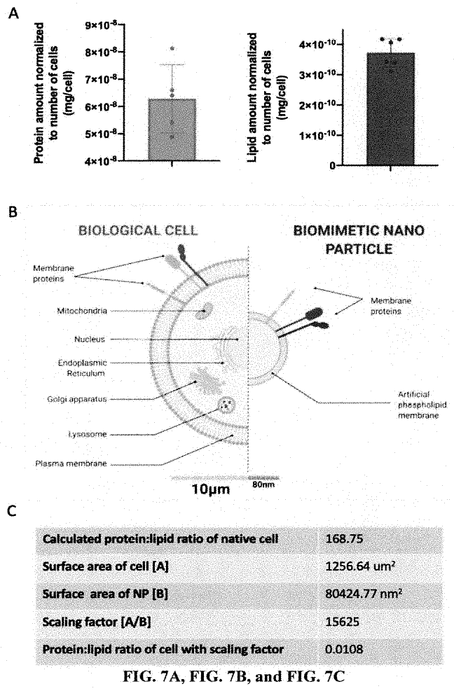

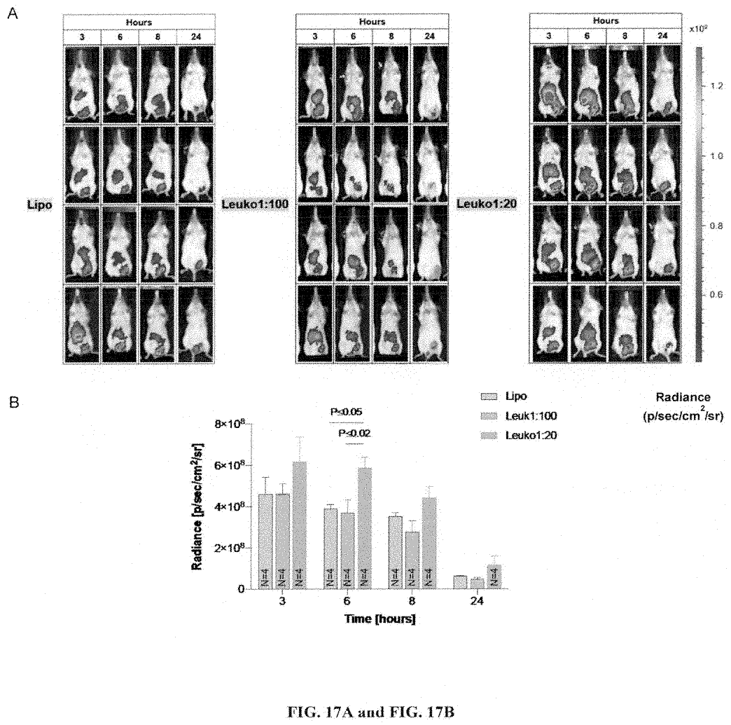

Effect of protein buffer on NP size (FIG. 6); Native protein:lipid ratio (FIG. 7A, FIG. 7B, and FIG. 7C). NP biomimetic markers, original WB membranes and quantification (FIG. 8A and FIG. 8B); CD11b orientation studies revealed equal distribution of these cytoplasmic and exoplasmic parts (FIG. 9A, FIG. 9B, and FIG. 9C); 21-day NP storage stability studies (FIG. 10A-FIG. 10E); Gating strategy for flow cytometry analysis of NP uptake (FIG. 11A and FIG. 11B.); Non-inflamed and LPS-inflamed endothelial cells (FIG. 12A and FIG. 12B); In vitro association and uptake of biomimetic NP by non-inflamed murine endothelial cells (FIG. 13A, FIG. 13B, and FIG. 13C); Percentage of positive cells in singlet gate of inflamed treated cells (FIG. 14); Ex vivo imaging of left and right ears for biomimetic NP targeting in a LLI model (FIG. 15A and FIG. 15B); TNBC tumor luminescence quantification (FIG. 16); In vivo biomimetic NP liver accumulation in TNBC model (FIG. 17A and FIG. 17B);

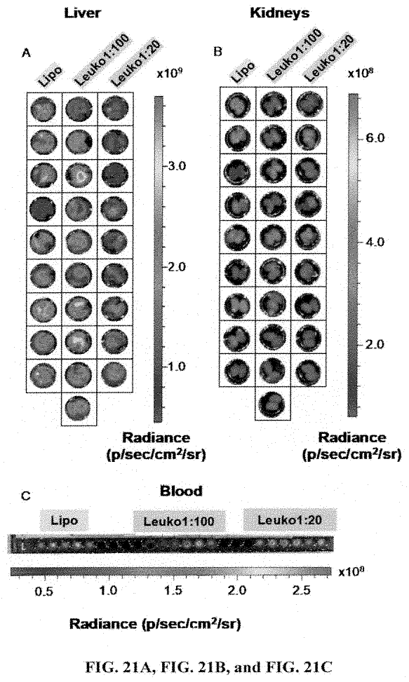

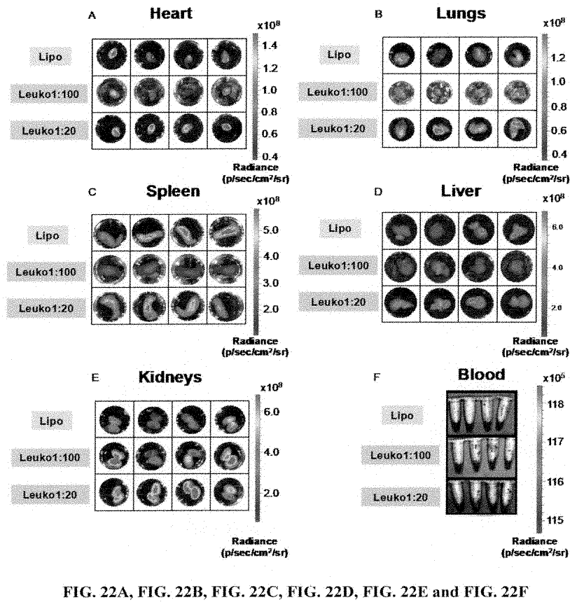

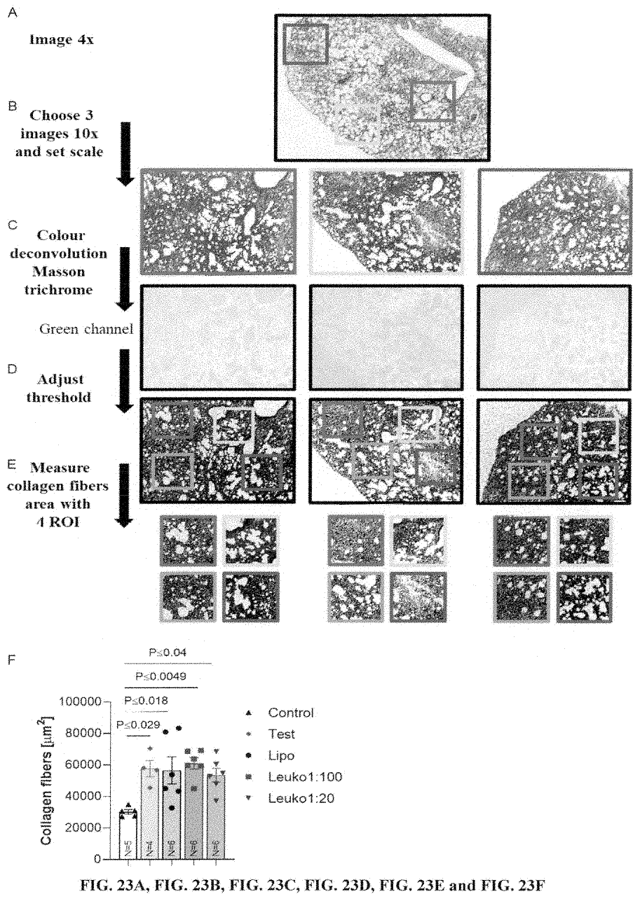

Ex vivo imaging of TNBC tumors for NP accumulation after 24 h (FIG. 18); Intravital microscopy for NP accumulation in inflamed vasculature of TNBC mice (FIG. 19A-FIG. 19E); Biomimetic NP accumulation in heart, lungs and spleen in LLI model after 8 h (FIG. 20A, FIG. 20B, and FIG. 20C); Biomimetic NP accumulation in liver, kidneys and blood in LLI model after 8 h (FIG. 21A, FIG. 21B, and FIG. 21C); Biomimetic NP accumulation in heart, lungs, spleen, liver, kidneys and blood of TNBC tumor-bearing mice after 24 h (FIG. 22A-FIG. 22F); Image analysis of collagen fibers workflow (FIG. 23A, FIG. 23B, FIG. 23C, FIG. 23D, FIG. 23E, and FIG. 23F).

### Results and Discussion

### Biodistribution and Safety of P:L Biomimetic NP

To further study the biodistribution of each biomimetic NP group and evaluate their potential as improved delivery vehicles, either 9-10 mice for the LLI model or 4 mice for the TNBC model, were injected with 100 μL of Cy5.5/Cy7-DSPE labeled NP. At the respective end timepoint (8 h for LLI and 24 h for TNBC), the heart, lungs, spleen, liver, kidneys, and blood were collected for fluorescence quantification (FIG. 20A-FIG. 20C, FIG. 21A-FIG. 21C, and FIG. 22A-FIG. 22F). The organs were collected, washed in PBS and then measured using IVIS. Upon euthanizing of the mice, blood was withdrawn from the hepatic vein.

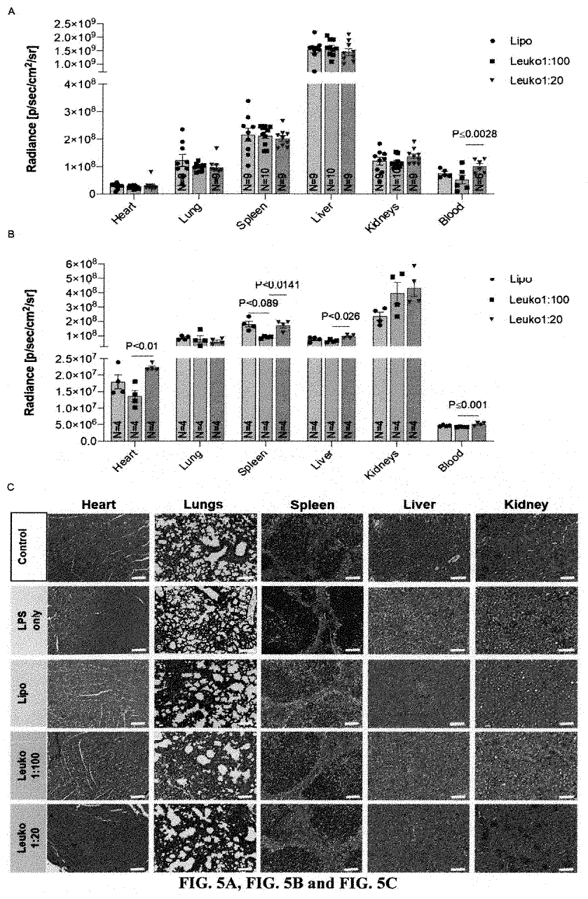

In the LLI model, the highest fluorescent signal among the collected organs was found in the liver followed by the spleen, lung, kidney, and heart (FIG. 5A). In contrast, for the TNBC model, the highest florescent signal among all the organs was measured in the kidney, spleen, liver, lung and heart (FIG. 5B). Interestingly, when compared to the Leuko1:100, higher fluorescent signal was measured in the blood of the Leuko1:20 group, both in the LLI and the TNBC models.

Finally, in order to assess if the different P:L biomimetic NP are well tolerated in vivo, tissue samples from the lungs, spleen, liver, kidneys and heart from the LLI mice were fixed, sectioned, and stained for hematoxylin and eosin (H&E). In addition, the lungs of these mice were stained for Masson's trichrome to assess the levels of fibrosis as an indication of inflammation. PBS injected mice and LPS-only injected mice served as controls and organs were processed in the same manner as previously described. No obvious injury was observed in any of organs from animals treated with P:L biomimetic NP (FIG. 5C). In addition, quantification of collagen fibers in the Masson's Trichrome stained lungs confirmed that the use of LPS did indeed induce inflammation, with no increase in fibrosis observed following treatment with biomimetic NP (FIG. 23A-FIG. 23F).

### Summary

In this Example, the effect of tuning the P:L ratio of leukocyte-based biomimetic NP was demonstrated on their physicochemical characteristics, biomimetic properties and biological functionality in targeting inflammation in vitro and in vivo. These leukocyte-based biomimetic NP were synthesized using a microfluidic-based, bottom-up approach, which aim to integrate leukocyte membrane proteins for improved targeting.¹¹ Recognizing the crucial role played by the membrane proteins' incorporation on the NP, we aimed to understand how tuning of the protein content affects key NP characteristics. Prior to tuning of this biomimetic NP formulation, key design parameters were set to ensure the maintenance of stringent properties for the future therapeutic applications. These key design criteria included the size, PDI, surface charge, retention of key leukocyte markers and 21-day stability after NP synthesis.

Upon successful synthesis of the NP with varying P:L ratios, we discovered key insights associated with the synthesis process. First and foremost, increasing the protein content on the NP did not affect the size, PDI or concentration of the particles. This finding was crucial in the efforts to maintain the fundamental characteristics that are known to contribute to the functional behavior of the particles. More importantly, unlike previously reported solid core biomimetic NP whose size increased after coated with cell membranes,10 no changes in NP were observed and validating the hypothesis that the proteins were incorporated into the lipid bilayer. Furthermore, increasing the protein amount did not negatively impact the structural integrity of the NP, as verified by cryo-TEM imaging.

On the contrary, if the extra proteins added in the aqueous phase of synthesis had not successfully integrated into the lipid bilayer, this would have been apparent both in the DLS size and PDI measurements. In particular, a heterogenous population of particles corresponding to a larger PDI would have been obtained.²⁹ Moreover, the decrease in zeta potential with decreasing P:L ratio was further confirmation of the successful integration of proteins into the lipid bilayer.

As more negatively charged proteins were incorporated, the surface charge became increasingly negative, as previously reported.¹⁷ In addition, the assumption that the hydrophilic core precludes access for hydrophobic membrane proteins together with removal of unbound membrane proteins from the NP after the synthesis by dialysis, further supports our claim that the extracted membrane proteins are integrated into the NP bilayer. Lastly, the protein buffer of the extracted membrane proteins was found to significantly impact the final size of the NP, a parameter which was determined to be kept less than 200 nm. By identifying the maximal volume of protein buffer that could be utilized in the synthesis, reproducibility of the NP formulation across batches was improved due to this key insight.

The presence and orientation of surface markers integrated into our biomimetic NP is imperative for their biological function (e.g., MPS evasion and inflammation targeting). SDS-PAGE confirmed the presence of more proteins with increasing P:L ratio and WB indicated the enrichment of key leukocyte markers on the NP, proteins that are known to dictate the innate behavior of these native immune cells. Markers of ‘self’, such as CD47 and CD45, enable these NP to delay clearance by components of the MPS and maintain longer circulation time.^{22,30}

On the other hand, CD18, CD11a and CD11b are proteins that mediate their ability to home to sites of inflammation, bind to the associated receptors on the inflamed endothelia and extravasate out into the surrounding tissue.^{31,32} While SDS-PAGE followed by WB for specific leukocyte membrane markers demonstrated the successful integration of proteins into the NP membrane, flow cytometry offered valuable insights on the orientation of these proteins. As a result of the NP self-assembly process and due to the fact that we had no engineered control over the way the membrane proteins would be integrated to the surface (e.g., cytoplasmic side of the membrane protein inside the NP and exoplasmic side outside the NP), the orientation of one leukocyte marker of interest (CD11b) known to be on our NP was studied (FIG. 9A, FIG. 9B, and FIG. 9C). Specifically, the data confirmed equal distribution between the integrated cytoplasmic and exoplasmic parts of CD11b among all the biomimetic NP groups. Despite this finding, higher association to inflamed endothelia was still assessed when the protein concentration was increased (Lipo<Leuko 1:100<Leuko 1:40<Leuko 1:20) supporting a claim of tunable targeting affect with respect to the protein concentration. As more of these proteins are enriched on the NP, we speculate that we improve the ability of the NP to reach the target site. Indeed, both of our in vitro and in vivo results validated this hypothesis.

Having confirmed the ability of our synthesis process to maintain the physicochemical and biomimetic characteristics of NP with varying P:L ratios, we were able to demonstrate the associated tuned NP behaviors. Recognizing that inflamed endothelial cells comprise the first populations of cells our NP encounter and interact with at the inflamed area in vivo, we confirmed the ability of our biomimetic NP to be taken up by these cells in vitro without inducing cytotoxicity. In fact, the uptake of the NP in this cell type followed a gradient pattern that correlated with increasing P:L ratio without any changes in the toxicity levels. This result suggested that both the targeting and internalization properties of the NP relied more on protein presence as opposed to other NP features, such as size, morphology and particle concentration. This was particularly evident in endothelial cells, which are not phagocytic cells like macrophages.³³ As a result, the endothelial cells showed very low NP signal in all of the groups, except the Leuko1:20. This observation aligns with the behavior of native leukocytes that use their membrane proteins, which includes the CD11b and CD18 found on our NP, to recognize sites of inflammations.^{31,32} Specifically, inflamed endothelial cells surrounding the site of inflammation upregulate proteins that mediate ligand-receptor interactions for leukocyte homing to the inflamed tissue.³⁴ It is believed that native NP behave in the same manner and confirmed this through the results obtained in both of the in vivo models.

The use of two different inflammation models allowed us to test the robustness of the NP targeting efficiencies under two disparate disease conditions—acute vs. chronic inflammatory response. While the in vivo models chosen for this study represent two different mechanisms of inflammation, the underlying mechanism of targeting for the NP remains the same—utilizing the integrated membrane proteins to specifically target the site of inflammation. As a result, the Leuko1:20 NP exhibited up to a 2.7-fold increase in accumulation to the site of inflammation when compared against liposomes (which do not contain any protein). The increased targeting to the inflamed vasculature with increasing protein content was further corroborated by IVM imaging. The arrival of NP to the target site improved with increasing P:L ratio, where Leuko1:20 demonstrated a 3-fold averaged increase in the accumulation with the lumen of the vessel. Taken together, these results validated the increased targeting efficiency associated with increasing the protein content on the NP, particularly in preferential accumulation to sites of inflammation.

While maintenance of key NP properties and enhancement of NP targeting efficiency was of utmost importance in this study, assessment of the effects of this tuning on healthy tissues was of equal importance. Although NP targeting emphasizes arrival at the target site, avoidance of organs that deter them arriving to that site must also be considered. Previous work has shown that 100 nm NP mostly accumulate in the liver and spleen,² a phenomena also seen in our in vivo imaging of the TNBC tumors. While the liposomes appeared to show decreased liver accumulation by 6 hr, Leuko1:20 exhibited liver accumulation even up to 8 hr. This suggests the longer circulation time of the Leuko1:20, which could be attributed to the higher presence of CD47 and CD45 on these NP. These cell markers might signal biological cues of “do not eat me” and “self” to the MPS. As these particles remained in circulation for a longer period, which was verified by the higher NP presence in the blood for both in vivo models, Leuko1:20 were also able to achieve improved tumor targeting. On the other hand, the Leuko1:100 contains less of these ‘self’ marker proteins which results in its reduced presence in the blood when compared to the Leuko1:20. In addition, our biodistribution results indicated that increasing the P:L ratio on the NP did not skew the particle accumulation to a different healthy organ when compared to the liposomes. In fact, accumulation profiles remained relatively similar across all the organs at the endpoint of organ collection. Therefore, we are able to minimize any unintended targeting to healthy organs. The safety profile of the NP was furthered corroborated by the histological analysis that showed no obvious signs of toxicity or increased lung fibrosis resulting from systemic administration of the NP. This absence of toxicity could be further explained by the use of naturally occurring membrane proteins on the NP which reduces the instigation of a foreign body response.

This work demonstrates a microfluidic approach that allows for the synthesis of reproducible NP as the P:L ratio of the desired biomimetic NP is tuned. The resulting leukocyte membrane protein integrated lipid NP was shown to retain the biological behavior of native leukocytes without affecting the NP physicochemical properties of size, PDI and concentration. In particular, these biomimetic NP demonstrated improved inflammation targeting and MPS evasion, a behavior that improved with increasing P:L ratio. The approach described in this paper highlights the importance of tuning key biomimetic NP synthesis parameters, especially those that directly dictate the biological properties of these NP. It is important to note that the protein extraction was a limiting factor in the maximum amount of protein that could be incorporated into the NP. Therefore, one aspect of future work aims to address this by methods to improve the protein extraction method and not be limited by the protein buffer in future formulations. Another aspect of future work will be to control the correct orientation of the membrane proteins during the NP fabrication. Finding a way to control this aspect of the assembly process will further improve the efficacy of these biomimetic NP.

To lay the foundations for future NP therapeutics using the disclosed P:L synthesis microfluidic methods, structural and biomimetic stability tests were performed after both short and long-term storage at 4° C. Both structural and biomimetic parameters remained constant over the 21-day test period which would allow researchers to store these kinds of NP for longer use. The work described here identifies a key discovery in the engineering of biomimetic NPs, which can be tuned for specific disease conditions using the body's own cells. The therapeutic benefits of these NP platforms can be further enhanced through loading of drugs or biological agents that treat the underlying disease condition. With an improved understanding of the relationships between synthesis parameters and the biological properties of biomimetic NP, future generations of NP hold even greater potential to target and treat specific diseases with greater efficacy and specificity.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used in this application, including the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, “bioactive” shall include a quality of a material such that the material has an osteointegrative potential, or in other words the ability to bond with bone. Generally, materials that are bioactive develop an adherent interface with tissues that resist substantial mechanical forces.

As used herein, a “biocompatible” material is a synthetic or natural material used to replace part of a living system or to function in intimate contact with living tissue. Biocompatible materials are intended to interface with biological systems to evaluate, treat, augment, or replace any tissue, organ, or function of the body. The biocompatible material has the ability to perform with an appropriate host response in a specific application and does not have toxic or injurious effects on biological systems. One example of a biocompatible material can be a biocompatible ceramic.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

As used herein, “chondrocyte” shall mean a differentiated cell responsible for secretion of extracellular matrix of cartilage. Preferably, the cells are from a compatible human donor. More preferably, the cells are from the patient (i.e., autologous cells).

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

As used herein, “fibroblast” shall mean a cell of connective tissue that secretes proteins and molecular collagen including fibrillar procollagen, fibronectin and collagenase, from which an extracellular fibrillar matrix of connective tissue may be formed. Fibroblasts synthesize and maintain the extracellular matrix of many tissues, including but not limited to connective tissue. The fibroblast cell may be mesodermally derived, and secrete proteins and molecular collagen including fibrillar procollagen, fibronectin and collagenase, from which an extracellular fibrillar matrix of connective tissue may be formed. A “fibroblast-like cell” means a cell that shares certain characteristics with a fibroblast (such as expression of certain proteins).

The terms “for example” or “e.g.,” as used herein, are used merely by way of example, without limitation intended, and should not be construed as referring only those items explicitly enumerated in the specification.

As used herein, “hard tissue” is intended to include mineralized tissues, such as bone, teeth, and cartilage. Mineralized tissues are biological tissues that incorporate minerals into soft matrices.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “implantable” or “suitable for implantation” means surgically appropriate for insertion into the body of a host, e.g., biocompatible, or having the desired design and physical properties.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

As used herein, “matrix” shall mean a three-dimensional structure fabricated with biomaterials. The biomaterials can be biologically-derived or synthetic.

As used herein, a “medical prosthetic device,” “medical implant,” “implant,” and such like, relate to a device intended to be implanted into the body of a vertebrate animal, such as a mammal, and in particular a human. Implants in the present context may be used to replace anatomy and/or restore any function of the body. Examples of such devices include, but are not limited to, dental implants and orthopedic implants. In the present context, orthopedic implants includes within its scope any device intended to be implanted into the body of a vertebrate animal, in particular a mammal such as a human, for preservation and restoration of the function of the musculoskeletal system, particularly joints and bones, including the alleviation of pain in these structures.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”), refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a patient may be any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, or any animal under the care of a veterinary or animal medical care practitioner.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

The term “pharmaceutically-acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gin), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

As used herein, “porosity” means the ratio of the volume of interstices of a material to a volume of a mass of the material.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about two to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, “soft tissue” is intended to include tissues that connect, support, or surround other structures and organs of the body, not being bone. Soft tissue includes ligaments, tendons, fascia, skin, fibrous tissues, fat, synovial membranes, epithelium, muscles, nerves and blood vessels.

As used herein, “stem cell” means an unspecialized cell that has the potential to develop into many different cell types in the body, such as mesenchymal osteoprogenitor cells, osteoblasts, osteocytes, osteoclasts, chondrocytes, and chondrocyte progenitor cells. Preferably, the cells are from a compatible human donor. More preferably, the cells are from the patient (i.e., autologous cells).

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The tern “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

### Biological Functional Equivalents

Modification and changes may be made in the structure of the nucleic acids, or to the vectors comprising them, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 1

| AMINO ACIDS | | CODONS | | | | | |
|---|---|---|---|---|---|---|---|
| Alanine | Ala | GCA | GCC | GCG | GCU | | |
| Cysteine | Cys | UGC | UGU | | | | |
| Aspartic acid | Asp | GAC | GAU | | | | |
| Glutamic acid | Glu | GAA | GAG | | | | |
| Phenylalanine | Phe | UUC | UUU | | | | |
| Glycine | Gly | GGA | GGC | GGG | GGU | | |
| Histidine | His | CAC | CAU | | | | |
| Isoleucine | Ile | AUA | AUC | AUU | | | |
| Lysine | Lys | AAA | AAG | | | | |
| Leucine | Leu | UUA | UUG | CUA | CUC | CUG | CUU |
| Methionine | Met | AUG | | | | | |
| Asparagine | Asn | AAC | AAU | | | | |
| Proline | Pro | CCA | CCC | CCG | CCU | | |
| Glutamine | Gln | CAA | CAG | | | | |
| Arginine | Arg | AGA | AGG | CGA | CGC | CGG | CGU |
| Serine | Ser | AGC | AGU | UCA | UCC | UCG | UCU |
| Threonine | Thr | ACA | ACC | ACG | ACU | | |
| Valine | Val | GUA | GUC | GUG | GUU | | |
| Tryptophan | Trp | UGG | | | | | |
| Tyrosine | Tyr | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### REFERENCES

The following references, to the extent that they provide exemplary procedural or other details supplementary to those set forth herein, are specifically incorporated herein by reference:
ACHOUR, A et al., Immunity, 9:199 (1998).
BERENDSEN, H B et al., J. Chem. Phys., 81:3684 (1984).
BLANCO, E et al., “Principles of nanoparticle design for overcoming biological barriers to drug delivery,” Nature Biotechnol., 33:941-951 (2015).
BUSSI, G et al., J Chem. Phys., 126:014101 (2007).
CAPRETTO, L el al., “Microfluidic and lab-on-a-chip preparation routes for organic nanoparticles and vesicular systems for nanomedicine applications,” Adv. Drug Del. Rev., 65:1496-1532 (2013).
CELIA, C et al., “Ethosomes® and Transfersomes® containing linoleic acid: physicochemical and technological features of topical drug delivery carriers for the potential treatment of melasma disorders,” Biomed. Microdevices, 14:119-130 (2012).
CORBO, C et al., ACS Nano., 11:3262 (2017).
CORBO, C et al., Biomater. Sci., (2017).
CORBO, C et al., Curr. Drug Targets, 16:1540 (2015).
CORBO, C et al., Int. J. Nanomed., 11:3049 (2016).
CORBO, C et al., Nanomedicine, 11:81 (2016).
CORBO, C et al., Nanoscale, (2017).
CORREIA, M G S et al., “Microfluidic manufacturing of phospholipid nanoparticles: stability, encapsulation efficacy, and drug release,” Int. J. Pharmaceut., (2016).
COSCO, D et al., “Aqueous-core PEG-coated PLA nanocapsules for an efficient entrapment of water soluble anticancer drugs and a smart therapeutic response,” Eur. J. Pharmaceut. Biopharmaceut., 89:30-39 (2015).
COSCO, D et al., Eur. J. Pharm. Biopharm., 89:30 (2015).
DAGUR, P K and J P MCCOY, Curr. Prot. Cytometry, 5.1. 1 (2015).
DE JONG, D H et al., J. Chem. Theory Comput., 9:687 (2012).
DEHAINI, D et al., Adv. Mater., (2017).
EVANGELOPOULOS, M et al., Biomaterials, 82:168 (2016).
FANG, R H et al., “Cancer cell membrane-coated nanoparticles for anticancer vaccination and drug delivery,” Nano Lett., 14:2181-2188 (2014).
GAGLIARDI, M, Ther. Deliv., 8:289 (2017).
GARG, S et al., J. Drug Target., 24:821 (2016).
GEBESHUBER, I C et al., “Nanomedicine and biomimetics: life sciences meet engineering & physics,” in ideas, Vol. 6-7 (2009).
HU, C-M J el al., “A biomimetic nanosponge that absorbs pore-forming toxins,” Nature Nanotechnol., 8:336-340 (2013).
HU, C-M J et al., “Erythrocyte membrane-camouflaged polymeric nanoparticles as a biomimetic delivery platform,” Proc. Nat'l. Acad. Sci. USA, 108:10980-10985 (2011).
HU, C-M J et al., “Nanoparticle biointerfacing by platelet membrane cloaking,” Nature, 526:118-121 (September 2015).
JAHN, A et al., “Controlled vesicle self-assembly in microfluidic channels with hydrodynamic focusing,”. Am. Chem. Society, 126:2674-2675 (2004).
JAHN, A el al., “Preparation of nanoparticles by continuous-flow microfluidics,” J. Nanopart. Res., 10:925-934 (2008).
JO, S et al., J. Comput. Chem., 29:1859 (2008).
KASTNER, E et al., “High-throughput manufacturing of size-tuned liposomes by a new microfluidics method using enhanced statistical tools for characterization,” Int. J. Pharmaceut., 477:361-368 (2014).
KASTNER, E et al., “Microfluidic-controlled manufacture of liposomes for the solubilisation of a poorly water soluble drug,” Int. J. Pharmaceut., 485:122-130 (2015).
LIU, D et al., Adv. Mater., 27:2298 (2015).
LIU, J et al., “Turbiscan Lab® Expert analysis of the biological demulsification of a water-in-oil emulsion by two biodemulsifiers,” J. Haz. Mat., 190:214-221 (2011).
LIU, J et al., J. Hazard. Mater., 190:214 (2011).
LUK, B T and ZHANG, L, “Cell membrane-camouflaged nanoparticles for drug delivery,” J. Contr. Rel., 220:600-607 (2015).
MARIANECCI, C el al., “Ammonium glycyrrhizinate-loaded niosomes as a potential nanotherapeutic system for anti-inflammatory activity in murine models,” Int. J. Nanomed., 9:635 (2014).
MARIANECCI, C et al., “Non-ionic surfactant vesicles in pulmonary glucocorticoid delivery: characterization and interaction with human lung fibroblasts,” J. Contr. Rel., 147:127-135 (2010).
MARRINK, S-J et al., J. Phys. Chem. B, 111:7812 (2007).
MAY, A et al., Bioinformatics, 30:326 (2014).
MEYER, RA el al., “Biomimetic particles as therapeutics,” Trends Biotechnol., 33:514-524 (2015).
MINARDI, S et al., “Evaluation of the osteoinductive potential of a bio-inspired scaffold mimicking the osteogenic niche, for bone augmentation,” Biomaterials, (2015).
MOLINARO, R et al., “Biomimetic proteolipid vesicles for targeting inflamed tissues,” Nature Materials, (2016).
MOLINARO, R et al., Current Medicinal Chem., 24:1 (2017).
MOLINARO, R et al., Nat. Mater., (2016).
MONOPOLI, M P et al., Nat. Nanotechnol., 7:779 (2012).
MONTICELLI, L et al., J. Chem. Theory Comput., 4:819 (2008).
PALOMBA, R et al., Sci. Rep., 6:34422 (2016).
PARODI, A et al., “Synthetic nanoparticles functionalized with biomimetic leukocyte membranes possess cell-like functions,” Nature Nanotechnol., 8:61-68 (2013).
PARODI, A et al., Biomaterials, 147:155 (2017).
PARODI, A et al., Nat. Nanotechnol., 8:61 (2013).
PATTNI, B S et al., “New developments in liposomal drug delivery,” Chem. Rev., 115:10938-10966 (2015).
PERRIE, Y et al., “Manufacturing Methods for Liposome Adjuvants. Vaccine Adjuvants: Methods and Protocols, 2016:127-144 (2016).
QI, Y et al., J. Chem. Theory Comput., 11:4486 (2015).
RAMACHANDRAN, S et al., “Cisplatin nanoliposomes for cancer therapy: AFM and fluorescence imaging of cisplatin encapsulation, stability, cellular uptake, and toxicity,” Langmuir, 22:8156-8162 (2006).
SALVATI, A et al., Nat. Nanotechnol., 8:137 (2013).
SHERMAN, M B et al., “Removal of divalent cations induces structural transitions in red clover necrotic mosaic virus, revealing a potential mechanism for RNA release,” J. Virol., 80:10395-10406 (2006).
STROOCK, A D et al., Science, 295:647 (2002).
SVENSON, S, Curr. Opin. Solid State Mater. Sci., 16:287 (2012).
VALENCIA, P M et al., “Microfluidic technologies for accelerating the clinical translation of nanoparticles,” Nature Nanotechnol., 7:623-629 (2012).
WALSH, C et al., “Microfluidic-based manufacture of siRNA-lipid nanoparticles for therapeutic applications,” Drug Del. Syst., 2014:109-120 (2014).
1. Patra, J. K.; Das, G.; Fraceto, L. F.; Campos, E. V. R.; Rodriguez-Torres, M. d. P.; Acosta-Torres, L. S.; Diaz-Torres, L. A.; Grillo, R.; Swamy, M. K.; Sharma, S.; Habtemariam, S.; Shin, H.-S., Nano Based Drug Delivery Systems: Recent Developments and Future Prospects. Journal of Nanobiotechnology 2018, 16, 71.
2. Blanco, E.; Shen, H.; Ferrari, M., Principles of Nanoparticle Design for Overcoming Biological Barriers to Drug Delivery. Nat. Biotechnol., 2015, 33, 941-951.
3. Alexis, F.; Pridgen, E.; Molnar, L. K.; Farokhzad, O. C., Factors Affecting the Clearance and Biodistribution of Polymeric Nanoparticles. Mol Pharm 2008, 5, 505-515.
4. Zinger, A.; Koren, L.; Adir, O.; Poley, M.; Alyan, M.; Yaari, Z.; Noor, N.; Krinsky, N.; Simon, A.; Gibori, H.; Krayem, M.; Mumblat, Y.; Kasten, S.; Ofir, S.; Fridman, E.; Milman, N.; Lubtow, M. M.; Liba, L.; Shklover, J.; Shainsky-Roitman, J. et al., Collagenase Nanoparticles Enhance the Penetration of Drugs into Pancreatic Tumors. ACS Nano 2019, 13, 11008-11021.
5. Pelaz, B.; del Pino, P.; Maffre, P.; Hartmann, R.; Gallego, M.; Rivera-Fernandez, S.; de la Fuente, J. M.; Nienhaus, G. U.; Parak, W. J., Surface Functionalization of Nanoparticles with Polyethylene Glycol: Effects on Protein Adsorption and Cellular Uptake. ACS Nano 2015, 9, 6996-7008.
6. Alkilany, A. M.; Zhu, L.; Weller, H.; Mews, A.; Parak, W. J.; Barz, M.; Feliu, N., Ligand Density on Nanoparticles: A Parameter with Critical Impact on Nanomedicine. Adv Drug Deliv Rev 2019, 143, 22-36.
7. Sroda, K.; Rydlewski, J.; Langner, M.; Kozubek, A.; Grzybek, M.; Sikorski, A. F., Repeated Injections of Peg-Pe Liposomes Generate Anti-Peg Antibodies. Cell Mol Biol Lett 2005, 10, 37-47.
8. Rodriguez, P. L.; Harada, T.; Christian, D. A.; Pantano, D. A.; Tsai, R. K.; Discher, D. E., Minimal “Self” Peptides That Inhibit Phagocytic Clearance and Enhance Delivery of Nanoparticles. Science 2013, 339, 971-975.
9. Zinger, A.; Brozovich, A.; Pasto, A.; Sushnitha, M.; Martinez, J. O.; Evangelopoulos, M.; Boada, C.; Tasciotti, E.; Taraballi, F., Bioinspired Extracellular Vesicles: Lessons Learned from Nature for Biomedicine and Bioengineering. Nanomaterials (Basel) 2020, 10, 2172.
10. Ben-Akiva, E.; Meyer, R. A.; Yu, H.; Smith, J. T.; Pardoll, D. M.; Green, J. J., Biomimetic Anisotropic Polymeric Nanoparticles Coated with Red Blood Cell Membranes for Enhanced Circulation and Toxin Removal. Science Advances 2020, 6, 9035.
11. Molinaro, R.; Martinez, J. O.; Zinger, A.; De Vita, A.; Storci, G.; Arrighetti, N.; De Rosa, E.; Hartman, K. A.; Basu, N.; Taghipour, N.; Corbo, C.; Tasciotti, E., Leukocyte-Mimicking Nanovesicles for Effective Doxorubicin Delivery to Treat Breast Cancer and Melanoma. Biomater Sci 2020, 8, 333-341.
12. Xie, W.; Deng, W. W.; Zan, M.; Rao, L.; Yu, G. T.; Zhu, D. M.; Wu, W. T.; Chen, B.; Ji, L. W.; Chen, L.; Liu, K.; Guo, S. S.; Huang, H. M.; Zhang, W. F.; Zhao, X.; Yuan, Y.; Dong, W.; Sun, Z. J.; Liu, W., Cancer Cell Membrane Camouflaged Nanoparticles to Realize Starvation Therapy Together with Checkpoint Blockades for Enhancing Cancer Therapy. ACS Nano 2019, 13, 2849-2857.
13. Parodi, A.; Molinaro, R.; Sushnitha, M.; Evangelopoulos, M.; Martinez, J. O.; Arrighetti, N.; Corbo, C.; Tasciotti, E., Bio-Inspired Engineering of Cell- and Virus-Like Nanoparticles for Drug Delivery. Biomaterials 2017, 147, 155-168.
14. Sushnitha, M.; Evangelopoulos, M.; Tasciotti, E.; Taraballi, F., Cell Membrane-Based Biomimetic Nanoparticles and the Immune System: Immunomodulatory Interactions to Therapeutic Applications. Frontiers in Bioengineering and Biotechnology 2020, 8, 627.
15. Hu, C. M.; Fang, R. H.; Copp, J.; Luk, B. T.; Zhang, L., A Biomimetic Nanosponge That Absorbs Pore-Forming Toxins. Nat Nanotechnol 2013, 8, 336-340.
16. Sun, H.; Su, J.; Meng, Q.; Yin, Q.; Chen, L.; Gu, W.; Zhang, P.; Zhang, Z.; Yu, H.; Wang, S.; Li, Y., Cancer-Cell-Biomimetic Nanoparticles for Targeted Therapy of Homotypic Tumors. Adv Mater 2016, 28, 9581-9588.
17. Molinaro, R.; Corbo, C.; Martinez, J. O.; Taraballi, F.; Evangelopoulos, M.; Minardi, S.; Yazdi, I. K.; Zhao, P.; De Rosa, E.; Sherman, M. B.; De Vita, A.; Toledano Furman, N. E.; Wang, X.; Parodi, A.; Tasciotti, E., Biomimetic Proteolipid Vesicles for Targeting Inflamed Tissues. Nat Mater 2016, 15, 1037-1046.
18. Zinger, A.; Baudo, G.; Naoi, T.; Giordano, F.; Lenna, S.; Massaro, M.; Ewing, A.; Kim, H. R.; Tasciotti, E.; Yustein, J. T.; Taraballi, F., Reproducible and Characterized Method for Ponatinib Encapsulation into Biomimetic Lipid Nanoparticles as a Platform for Multi-Tyrosine Kinase-Targeted Therapy. ACS Applied Bio Materials 2020, 3, 6737-6745.
19. Molinaro, R.; Evangelopoulos, M.; Hoffman, J. R.; Corbo, C.; Taraballi, F.; Martinez, J. O.; Hartman, K. A.; Cosco, D.; Costa, G.; Romeo, I.; Sherman, M.; Paolino, D.; Alcaro, S.; Tasciotti, E., Design and Development of Biomimetic Nanovesicles Using a Microfluidic Approach. Adv Mater 2018, 30, e1702749.
20. Zook, J. M.; Vreeland, W. N., Effects of Temperature, Acyl Chain Length, and Flow-Rate Ratio on Liposome Formation and Size in a Microfluidic Hydrodynamic Focusing Device. Soft Matter 2010, 6, 1352-1360.
21. Dimov, N.; Kastner, E.; Hussain, M.; Perrie, Y.; Szita, N., Formation and Purification of Tailored Liposomes for Drug Delivery Using a Module-Based Micro Continuous-Flow System. Sci Rep 2017, 7, 12045.
22. Martinez, J. O.; Molinaro, R.; Hartman, K. A.; Boada, C.; Sukhovershin, R.; De Rosa, E.; Kirui, D.; Zhang, S.; Evangelopoulos, M.; Carter, A. M.; Bibb, J. A.; Cooke, J. P.; Tasciotti, E., Biomimetic Nanoparticles with Enhanced Affinity Towards Activated Endothelium as Versatile Tools for Theranostic Drug Delivery. Theranostics 2018, 8, 1131-1145.
23. Dudley, A. C., Tumor Endothelial Cells. Cold Spring Harb Perspect Med 2012, 2, a006536.
24. Schouten, M.; Wiersinga, W. J.; Levi, M.; van der Poll, T., Inflammation, Endothelium, and Coagulation in Sepsis. J Leukoc Biol 2008, 83, 536-545.
25. Stanimirovic, D.; Satoh, K., Inflammatory Mediators of Cerebral Endothelium: A Role in Ischemic Brain Inflammation. Brain Pathol 2000, 10, 113-126.
26. Gimbrone, M. A., Jr.; Garcia-Cardena, G., Endothelial Cell Dysfunction and the Pathobiology of Atherosclerosis. Circ Res 2016, 118, 620-636.
27. Nourshargh, S.; Alon, R., Leukocyte Migration into Inflamed Tissues. Immunity 2014, 41, 694-707.
28. Biinger, S.; Roblick, U. J.; Habermann, J. K., Comparison of Five Commercial Extraction Kits for Subsequent Membrane Protein Profiling. Cytotechnology 2009, 61, 153-159.
29. Danaei, M.; Dehghankhold, M.; Ataei, S.; Hasanzadeh Davarani, F.; Javanmard, R.; Dokhani, A.; Khorasani, S.; Mozafari, M. R., Impact of Particle Size and Polydispersity Index on the Clinical Applications of Lipidic Nanocarrier Systems. Pharmaceutics 2018, 10, 57.
30. Rodriguez, P. L.; Harada, T.; Christian, D. A.; Pantano, D. A.; Tsai, R. K.; Discher, D. E., Minimal “Self” Peptides That Inhibit Phagocytic Clearance and Enhance Delivery of Nanoparticles. Science 2013, 339, 971-975.
31. Ley, K., Molecular Mechanisms of Leukocyte Recruitment in the Inflammatory Process. Cardiovasc Res 1996, 32, 733-742.
32. Ley, K.; Laudanna, C.; Cybulsky, M. I.; Nourshargh, S., Getting to the Site of Inflammation: The Leukocyte Adhesion Cascade Updated. Nat Rev Immunol 2007, 7, 678-689.
33. Arango Duque, G.; Descoteaux, A., Macrophages Tell the Non-Professionals What to Do. Dev Cell 2016, 39, 633-635.
34. Vestweber, D., How Leukocytes Cross the Vascular Endothelium. Nat Rev Immunol 2015, 15, 692-704.

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are specifically incorporated herein by reference to the same extent as if each reference was individually and specifically indicated to be incorporated by reference, and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing,” with reference to an element or elements is intended to provide support for a similar aspect or embodiment of the invention that “consists of,” “consists essentially of,” or “substantially comprises” the particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and methods and in the steps or in the sequence of steps of the method described herein without departing from the concept, spirit and scope of the invention. More specifically, it will be apparent that certain agents that are chemically- or physiologically-related may be substituted for the agents described herein while the same or similar results would be achieved. All such similar substitutes and modifications apparent to those ordinarily skilled in the art are deemed to be within the spirit, scope, and concept of the invention as defined by the appended claims.

What is claimed is:

1. A method of preparing a population of biomimetic proteolipid nanovesicles composed of synthetic phospholipids and cholesterol, enriched of leukocyte membrane fragments, and surrounding an aqueous core, the method comprising:
a) dissolving predetermined amounts of two or more selected phosphocholine-based phospholipids, and cholesterol in ethanol to a final lipid concentration of approximately 9 mM to produce an organic lipid solution;
b) dissolving a predetermined amount of at least one selected membrane protein in water to produce an aqueous protein solution;
c) loading the organic lipid solution of (a) into the organic phase inlet of a microfluidic mixer, and loading the aqueous protein solution of (b) into the aqueous phase inlet of the microfluidic mixer, wherein the microfluidic mixer is set to a predetermined reaction temperature; and
d) adjusting the flow rates of each inlet stream and the flow ratio between each of the inlet streams of the microfluidic mixer to produce the population of biomimetic proteolipid nanovesicles therefrom.

2. The method of claim 1, wherein adjustment of the flow rates or the flow ratio results in the production of a population of biomimetic proteolipid nanovesicles having a desired property selected from the group consisting of a consistent size, a consistent size homogeneity, a selected amount of protein incorporation into the membrane fragments, nanovesicle stability, and any combination thereof.

3. The method of claim 1, further comprising purifying the population of biomimetic proteolipid nanovesicles so produced by dialysis, ultracentrifugation, or a combination thereof.

4. The method of claim 1, wherein the selected flow rate is 1 mL/min, the selected flow ratio is 2:1 (organic phase-to-aqueous phase), and the predetermined reaction temperature is approximately 45° C.

5. The method of claim 5, wherein the efficiency of protein incorporation into the leukocyte membrane fragments is at least 40% to 60% higher than that of biosimilar nanovesicles prepared by conventional thin-layer evaporation.

6. The method of claim 1, wherein the total number of nanovesicles produced per gram of lipid is at least 100% higher than that of biosimilar nanovesicles prepared by conventional thin-layer evaporation.

7. The method of claim 6, wherein the total number of nanovesicles produced per gram of lipid is at least 200% higher than that of biosimilar nanovesicles prepared by conventional thin-layer evaporation.

8. The method of claim 1, wherein the efficiency of protein incorporation into the membrane fragments is at least 10-fold higher than for biosimilar nanovesicles prepared by conventional thin-layer evaporation.

9. The method of claim 8, wherein the efficiency of protein incorporation into the membrane fragments is at least 20-fold higher than for biosimilar nanovesicles prepared by conventional thin-layer evaporation.

10. The method of claim 1, wherein the proteolipid nanovesicles are about 100 to about 1000 nm in average diameter.

11. The method of claim 1, wherein the selected phosphocholine-based phospholipids are selected from the group consisting of phosphatidylcholine, egg phosphatidic acid, 1,2-dioleoyl-sn-glycerophosphocholine (DOPC), 1,2-diolcoyl-sn-glycerophosphoethanolamine (DOPE), 1,2-dipalmitoyl-sn-glycerophosphocholine (DPPC), 1,2-distearoyl-sn-glycerophosphocholine (DSPC), and any combination thereof.

12. The method of claim 1, wherein the resulting population of biomimetic proteolipid nanovesicles are stable in solution at 4° C. for about two to about three weeks.

13. The method of claim 1, wherein the leukocyte membrane fragments are derived from leukocyte plasma membranes.

14. The method of claim 13, wherein the leukocyte membrane fragments are derived from human leukocyte plasma membranes.

15. The method of claim 1, wherein the protein-to-lipid ratio is from about 1:10 (wt./wt.) to about 1:40 (wt./wt.).

16. The method of claim 15, wherein the protein-to-lipid ratio is from about 1:15 (wt./wt.) to about 1:30 (wt./wt.).

17. The method of claim 16, wherein the protein-to-lipid ratio is from about 1:20 (wt./wt.) to about 1:25 (wt./wt.).

18. The method of claim 1, wherein the resulting population of biomimetic proteolipid nanovesicles further comprises a diagnostic or therapeutic agent, or a combination thereof.

19. A population of biomimetic proteolipid nanovesicles that comprise one or more cell membrane proteins that confer the natural tropism of a leukocyte to the nanovesicles, and wherein specific tuning of the integration of one or more polypeptides: a) affects the in vivo behavior of the resulting nanovesicles; b) alters the efficacy of, distribution of, or the targeting of the nanovesicles in one or more particular mammalian cell types; or any combination thereof.

20. The population of biomimetic proteolipid nanovesicles of claim 19, wherein specific tuning of the integration of one or more polypeptides improves the targeting of the nanovesicles to mammalian endothelial cells.

21. The population of biomimetic proteolipid nanovesicles of claim 20, wherein the mammalian endothelial cells are inflamed.

22. The population of biomimetic proteolipid nanovesicles of claim 19, wherein the protein-to-lipid ratio is from about 1:15 (wt./wt.) to about 1:30 (wt./wt.).

23. The population of biomimetic proteolipid nanovesicles of claim 22, wherein the protein-to-lipid ratio is from about 1:20 (wt./wt.) to about 1:25 (wt./wt.).

24. The population of biomimetic proteolipid nanovesicles of claim 23, wherein the protein-to-lipid ratio is about 1:20 (wt./wt.).

25. A drug delivery composition comprising the population of biomimetic proteolipid nanovesicles of claim 19, and a pharmaceutical excipient, buffer, diluent, or any combination thereof.

26. The drug delivery composition of claim 25, further comprising at least one therapeutic agent selected from the group consisting of a chemotherapeutic drug, an antibiotic, an anesthetic, an analgesic, an anti-inflammatory, a small molecule, an immune-stimulating agent, a tumor growth inhibitor, a protein, a peptide, an RNA molecule, a DNA molecule, an siRNA molecule, a RNAi molecule, a ssRNA molecule, a growth factor, an enzyme inhibitor, a binding protein, a blocking peptide, and any combination thereof.

27. The drug delivery composition of claim 26, wherein the proteolipid nanovesicles are adapted and configured to release the at least one therapeutic agent in response to an external stimulus, in response to a change in the environment of the population of biomimetic proteolipid nanovesicles, or as a result of degradation of the proteolipid nanovesicles.

28. The drug delivery composition of claim 25, wherein degradation of the population of biomimetic proteolipid nanovesicles occurs via enzyme-facilitated biodegradation of one or more of the phospholipids or the cholesterol comprising them.

29. The drug delivery composition of claim 25, wherein the leukocyte membrane fragments comprise at least one cellular-targeting moiety.

30. The drug delivery composition of claim 29, wherein the at least one cellular-targeting moiety is selected from the group consisting of a chemically-targeting moiety, a physically-targeting moiety, a geometrically-targeting moiety, a ligand, a ligand-binding moiety, a receptor, a receptor-binding moiety, an antibody or an antigen-binding fragment thereof, and any combination thereof.

31. The drug delivery composition of claim 30, wherein the at least a first cellular-targeting moiety comprises a plurality of distinct antigenic ligands that elicit one or more target-specific immune responses in a mammalian host cell that is contacted with the population of nanovesicles.

32. The drug delivery composition of claim 25, further comprising a diagnostic agent.

33. The drug delivery composition of claim 32, wherein the diagnostic reagent is selected from the group consisting of an imaging agent, a contrast agent, a fluorescent label, a radiolabel, a magnetic resonance imaging label, a spin label, and any combination thereof.

34. The drug delivery composition of claim 25, comprising a chemically-targeting moiety that is disposed on the surface of the proteolipid nanovesicles, and that comprises a ligand, a dendrimer, an oligomer, an aptamer, a binding protein, an antibody, an antigen-binding fragment thereof, a biomolecule, or any combination thereof.

35. A population of isolated mammalian cells comprising the population of biomimetic proteolipid nanovesicles of claim 19.

36. A pharmaceutical formulation comprising the population of biomimetic proteolipid nanovesicles of claim 19, and a pharmaceutically-acceptable buffer, diluent, excipient, or vehicle, wherein the protein-to-lipid ratio of the nanovesicles is about 1:20 (wt./wt.).

37. A kit comprising the pharmaceutical formulation of claim 36, and instructions for administering the composition to a mammal in need thereof.

38. A method for providing one or more active agents to a population of cells within the body of an animal, comprising administering to the animal an amount of the pharmaceutical formulation of claim 36, for a time effective to provide the one or more active agents to the population of cells within the body of the animal.

39. The method of claim 38, wherein the animal is at risk for developing, is suspected of having, or is diagnosed with inflammation, a solid tumor, a cancer, or any combination thereof.

40. A method of targeting a diagnostic, therapeutic, or prophylactic agent to one or more inflamed sites within the body of a mammalian subject in need thereof, comprising administering to the subject an effective amount of the pharmaceutical formulation of claim 36.

41. The method of claim 40, wherein the therapeutic agent further comprises at least a first chemotherapeutic agent.

42. The method of claim 40, wherein accumulation of the nanovesicles is at least 8- to 13-fold higher in the inflamed site as compared to non-inflamed tissues when the formulation is administered systemically to the mammal.
